(19) United States
(12) Patent Application Publication
(10) Pub. No.: US 2019/0225702 A1
(43) Pub. Date: Jul. 25, 2019

(54) INNATE IMMUNE CELL TRISPECIFIC BINDING PROTEINS AND METHODS OF USE

(71) Applicant: Harpoon Therapeutics, Inc., South San Francisco, CA (US)

(72) Inventors: Patrick BAEUERLE, Gauting (DE); Luke EVNIN, San Francisco, CA (US); Jeanmarie GUENOT, San Francisco, CA (US); Holger WESCHE, South San Francisco, CA (US)

(21) Appl. No.: 16/339,263

(22) PCT Filed: Oct. 13, 2017
(86) PCT No.: PCT/US17/56530

§ 371 Date: Apr. 3, 2019

Related U.S. Application Data
(60) Provisional application No. 62/408,601, filed on Oct. 14, 2016.

(51) Int. Cl. C07K 16/28; A61P 35/00

(52) U.S. Cl. CPC C07K 16/2887; C07K 16/283; A61P 35/00; C07K 2317/94; C07K 2317/622; C07K 2317/33; C07K 2317/73; C07K 2317/31

(57) ABSTRACT

Provided herein are trispecific antigen-binding proteins comprising a domain binding to innate immune cells, a half-life extension domain, and a domain binding to a target antigen. Also provided are pharmaceutical compositions thereof, as well as nucleic acids, recombinant expression vectors and host cells for making such trispecific antigen-binding proteins. Also disclosed are methods of using the disclosed trispecific antigen-binding proteins in the prevention, and/or treatment diseases, conditions and disorders.

### CROSS-REFERENCE

This application claims the benefit of U.S. Provisional Application No. 62/408,601 filed Oct. 14, 2016, which is incorporated by reference herein in its entirety.

### BACKGROUND OF THE INVENTION

The selective destruction of an individual cell or a specific cell type is often desirable in a variety of clinical settings. For example, it is a primary goal of cancer therapy to specifically destroy tumor cells, while leaving healthy cells and tissues intact and undamaged. One such method is by inducing an immune response against the tumor, to make innate immune cells such as natural killer (NK) cells or dendritic cells attack and destroy tumor cells.

### SUMMARY OF THE INVENTION

Provided herein are trispecific antigen-binding protein, pharmaceutical compositions thereof, as nucleic acids, recombinant expression vectors and host cells for making such trispecific antigen-binding proteins, and methods of use for the treatment of diseases, disorders, or conditions. In one aspect, described herein are trispecific antigen-binding proteins wherein said proteins comprise (a) a first domain (A) which specifically binds to a human innate immune cell; (b) a second domain (B) which is a half-life extension domain; and (c) a third domain (C) which specifically binds to a target antigen, wherein the domains are linked in the order H₂N-(A)-(B)-(C)-COOH, H₂N-(A)-(C)-(B)-COOH, H₂N-(B)-(A)-(C)-COOH, H₂N-(B)-(C)-(A)-COOH, H₂N-(C)-(B)-(A)-COOH, or H₂N-(C)-(A)-(B)-COOH by linkers L1 and L2.

Also provided herein in certain aspects are trispecific antigen-binding proteins, wherein said proteins comprise (a) a first domain (A) which specifically binds to a human innate immune cell; (b) a second domain (B) which is a half-life extension domain; and (c) a third domain (C) which specifically binds to a target antigen, wherein the domains are linked in the order H₂N-(A)-(C)-(B)-COOH, H₂N-(B)-(A)-(C)-COOH, H₂N-(C)-(B)-(A)-COOH, or by linkers L1 and L2.

Also provided herein in certain aspects are trispecific antigen-binding proteins, wherein said proteins comprise (a) a first domain (A) which specifically binds to a human innate immune cell; (b) a second domain (B) which is a half-life extension domain; and (c) a third domain (C) which specifically binds to a target antigen, wherein the domains are linked in the order H₂N-(A)-(B)-(C)-COOH, H₂N-(A)-(C)-(B)-COOH, H₂N-(B)-(A)-(C)-COOH, H₂N-(B)-(C)-(A)-COOH, H₂N-(C)-(B)-(A)-COOH, or H₂N-(C)-(A)-(B)-COOH by linkers L1 and L2, and wherein the first domain binds to a human innate immune cell with a KD of greater than 100 nM.

Also provided herein in certain aspects are trispecific antigen-binding proteins, wherein said proteins comprise (a) a first domain (A) which specifically binds to a human innate immune cell; (b) a second domain (B) which is a half-life extension domain; and (c) a third domain (C) which specifically binds to a target antigen, wherein the domains are linked in the order H₂N-(A)-(B)-(C)-COOH, H₂N-(A)-(C)-(B)-COOH, H₂N-(B)-(A)-(C)-COOH, H₂N-(B)-(C)-(A)-COOH, H₂N-(C)-(B)-(A)-COOH, or H₂N-(C)-(A)-(B)-COOH by linkers L1 and L2, and wherein the protein has a molecular weight of less than 55 kDa.

Also provided herein in certain aspects are trispecific antigen-binding proteins, wherein said proteins comprise (a) a first domain (A) which specifically binds to a human innate immune cell; (b) a second domain (B) which is a half-life extension domain; and (c) a third domain (C) which specifically binds to a target antigen, wherein the domains are linked in the order H₂N-(A)-(B)-(C)-COOH, H₂N-(A)-(C)-(B)-COOH, H₂N-(B)-(A)-(C)-COOH, H₂N-(B)-(C)-(A)-COOH, H₂N-(C)-(B)-(A)-COOH, or H₂N-(C)-(A)-(B)-COOH by linkers L1 and L2, and wherein B comprises a single domain antibody that binds to serum albumin.

Various embodiments of trispecific antigen-binding proteins are also provided herein, contemplated for any aspect herein, alone or in combination. In some embodiments, first domain comprises a variable light chain and variable heavy chain each of which is capable of specifically binding to a human innate immune cell. In some embodiments, the variable light chain is a λ (lamda) light chain. In some embodiments, the variable light chain is a κ (kappa) light chain. In some embodiments, the first domain comprises a single-chain variable fragment (scFv) specific to a human innate immune cell. In some embodiments, the first domain is specific for a dendritic cell. In some embodiments, the first domain is specific for a plasmacytoid dendritic cell. In some embodiments, the first domain is specific for a natural killer cell. In some embodiments, the first domain is specific for a natural killer T cell. In some embodiments, the first domain is specific for a monocyte. In some embodiments, the first domain is specific for a neutrophil. In some embodiments, the first domain is specific for an eosinophil. In some embodiments, the first domain is specific for a basophil immune cell. In some embodiments, the first domain comprises complementary determining regions (CDRs) selected from the group consisting of Lorvotuzumab, 3C12C, CSL362, 3G8, rMil2, E4, NNC141-0100. In some embodiments, the first domain is humanized or human. In some embodiments, the first domain has a KD binding of 1000 nM or less to a cell surface marker on innate immune cells. In some embodiments, the first domain has a KD binding of 100 nM or less to a cell surface marker on innate immune cells. In some embodiments, the first domain has a KD binding of 10 nM or less to a cell surface marker on innate immune cells. In some embodiments, the first domain has crossreactivity with a cynomolgus innate immune cell. In some embodiments, the first domain comprises an amino acid sequence provided herein.

In some embodiments, the second domain binds human serum albumin. In some embodiments, the second domain comprises a scFv, a variable heavy domain (VH), a variable light domain (VL), a single domain antibody, a peptide, a ligand, or a small molecule. In some embodiments, the second domain comprises a scFv. In some embodiments, the second domain comprises a VH domain. In some embodiments, the second domain comprises a VL domain. In some embodiments, the second domain comprises a single domain antibody. In some embodiments, the second domain comprises a peptide. In some embodiments, the second domain comprises a ligand. In some embodiments, the second domain comprises a small molecule entity.

In some embodiments, the third domain comprises a scFv, a VH domain, a VL domain, a non-Ig domain, a ligand, a knottin, or a small molecule entity that specifically binds to a target antigen. In some embodiments, the third domain is specific to a cell surface molecule. In some embodiments, the third domain is specific to a tumor antigen.

In some embodiments, linkers L1 and L2 are peptide linkers. In some embodiments, linkers L1 and L2 independently consist of about 20 or less amino acid residues. In some embodiments, linkers L1 and L2 are each independently selected from (GS)n (SEQ ID NO: 49), (GGS)n (SEQ ID NO: 50), (GGGS)n (SEQ ID NO: 51), (GGSG)n (SEQ ID NO: 52), (GGSGG)n (SEQ ID NO: 53), or (GGGGS)n (SEQ ID NO: 54), wherein n is 1, 2, 3, 4, 5, 6, 7, 8, 9, or 10. In some embodiments, linkers L1 and L2 are each independently (GGGGS)4 (SEQ ID NO: 55) or (GGGGS)3 (SEQ ID NO: 56). In some embodiments, linkers L1 and L2 are chemical linkers.

In some embodiments, the first domain is at the N-terminus of the protein. In some embodiments, the second domain is at the N-terminus of the protein. In some embodiments, the third domain is at the N-terminus of the protein. In some embodiments, the first domain is at the C-terminus of the protein. In some embodiments, the second domain is at the C-terminus of the protein. In some embodiments, the third domain is at the C-terminus of the protein.

In some embodiments, the protein is less than about 80 kDa. In some embodiments, the protein is about 50 to about 75 kDa. In some embodiments, the protein is less than about 50 kDa. In some embodiments, the protein is less than about 40 kDa. In some embodiments, the protein is about 20 to about 40 kDa. In some embodiments, the protein has an elimination half-time of at least about 50 hours. In some embodiments, the protein has an elimination half-time of at least about 100 hours. In some embodiments, the protein has increased tissue penetration as compared to an IgG to the same target antigen.

Also provided herein, in another aspect are polynucleotides encoding trispecific antigen-binding proteins according to any one of the above embodiments. In another aspect provided herein are vectors comprising the described polynucleotides. In another aspect, provided herein are host cells transformed with the described vectors

In yet another aspect, provided herein are pharmaceutical compositions comprising a trispecific antigen-binding protein of any of the above embodiments, a polynucleotide encoding a trispecific antigen-binding protein of any of the above embodiments, a vector comprising the described polynucleotides, or a host cell transformed with a vector of any of the above embodiments and a pharmaceutically acceptable carrier.

Also provided herein, are processes for the production of trispecific antigen-binding proteins according to any of the aspects and embodiments herein, said process comprising culturing a host transformed or transfected with a vector comprising a nucleic acid sequence encoding any trispecific antigen-binding protein herein under conditions allowing the expression of the protein and recovering and purifying the produced protein from the culture.

Also provided herein are methods for the treatment amelioration of a proliferative disease, a tumorous disease, an inflammatory disease, an immunological disorder, an autoimmune disease, an infectious disease, viral disease, allergic reactions, parasitic reactions, graft-versus-host diseases or host-versus-graft diseases comprising the administration of a trispecific antigen-binding protein of any of the above embodiments to a subject in need of such a treatment or amelioration. In some embodiments, the subject is a human. In some embodiments, the method further comprises administration of an agent in combination with the trispecific antigen-binding protein described herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is schematic representation of an exemplary trispecific antigen-binding protein where the protein has an constant core element comprising an anti-CD16 single chain variable fragment (scFv) and an anti-HSA variable heavy chain region; and a variable target binding domain that can be a VH, scFv, a non-Ig binder, or ligand.

FIG. 2 is schematic representation of additional exemplary trispecific antigen-binding proteins constructed for optimal tissue penetration. FIG. 2 left, an exemplary trispecific antigen-binding protein comprising single domain antibody fragments for all its domains. FIG. 2 middle, an exemplary trispecific antigen-binding protein comprising a knottin that binds to a target antigen. FIG. 2 right, an exemplary trispecific antigen-binding protein comprising a natural ligand that binds to a target antigen.

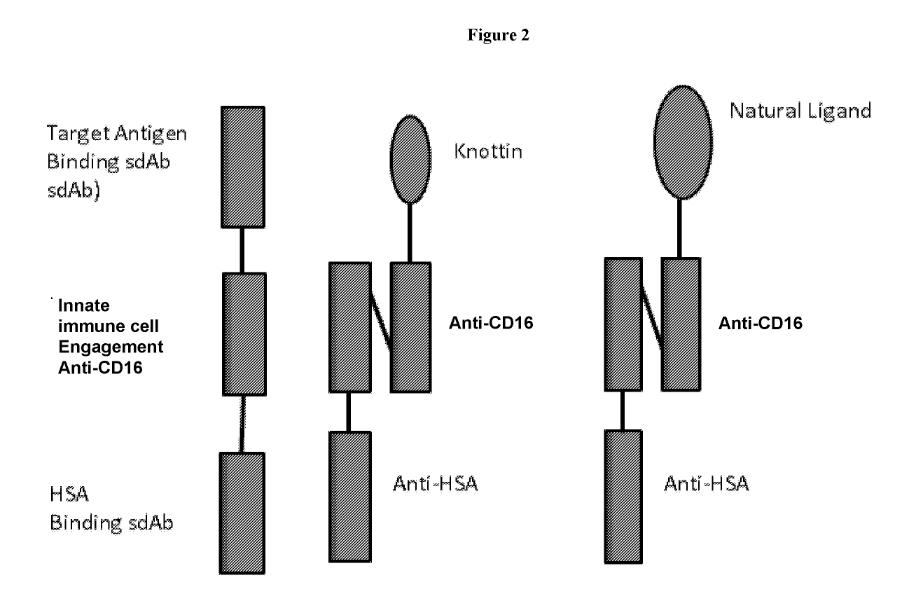

FIG. 3 is a schematic representation of attaching a small molecule entity binder to a trispecific antigen-binding protein. The trispecific antigen-binding protein comprises a sortase recognition sequence as its target antigen binding domain. Upon incubating the protein with a sortase and a glycine-attached small molecule binder, the sortase ligates or conjugates the small molecule binder onto the recognition site. Figure discloses “LPETGG” as SEQ ID NO: 51 and “LPETG” as SEQ ID NO: 48.

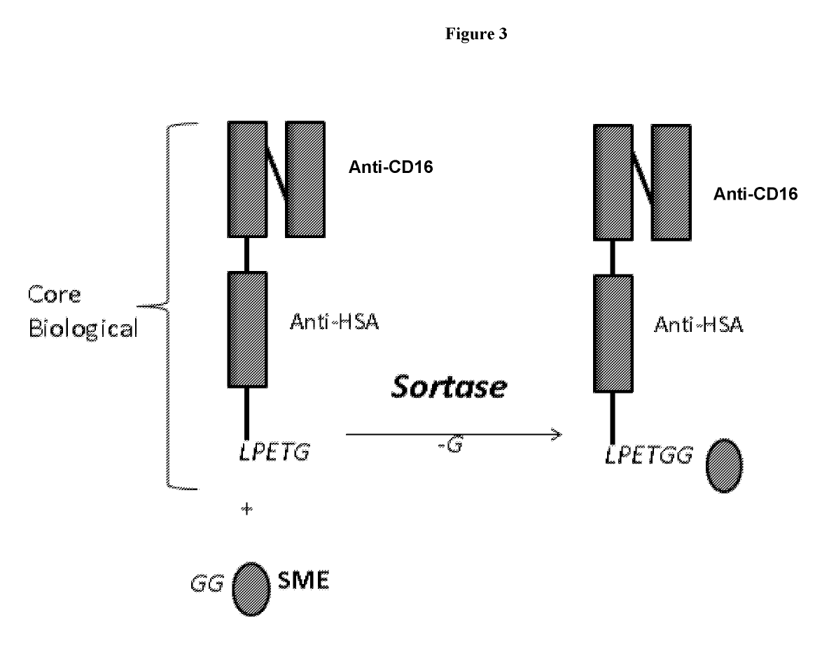

FIG. 4 is schematic representation of the six different ways in which the three domains of these trispecific antigen binding molecules can be arranged.

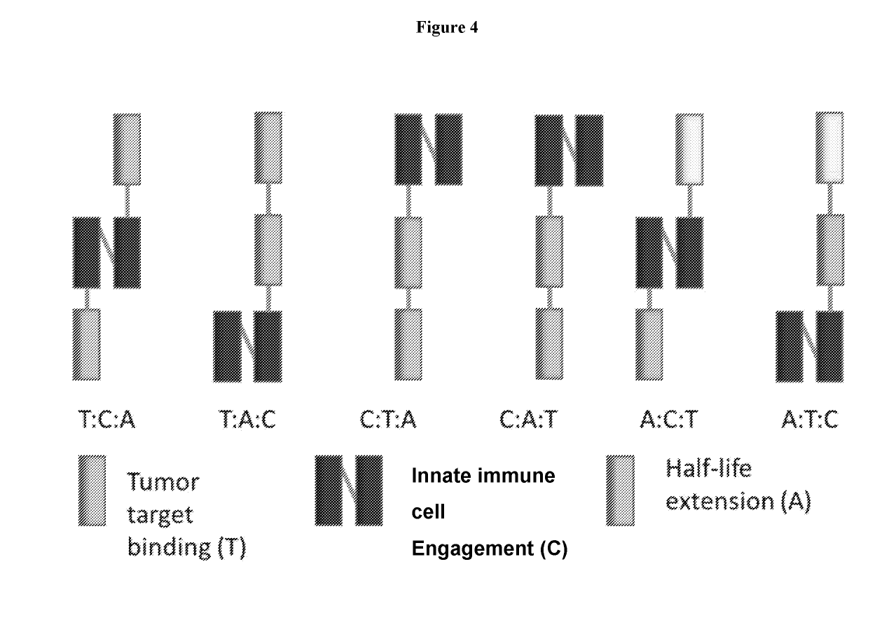

### DETAILED DESCRIPTION OF THE INVENTION

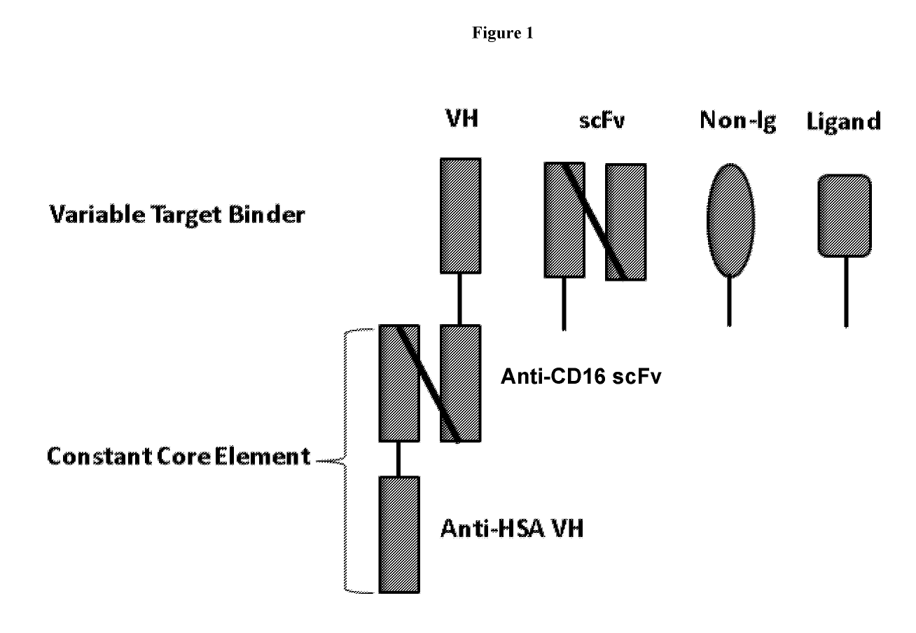

Described herein are trispecific antigen-binding proteins, pharmaceutical compositions thereof, as well as nucleic acids, recombinant expression vectors and host cells for making such trispecific antigen-binding proteins. Also provided are methods of using the disclosed trispecific antigen-binding proteins in the prevention, and/or treatment of diseases, conditions and disorders. The trispecific antigen-binding proteins are capable of specifically binding to a target antigen as well as an innate immune cell and a half-life extension domain, such as a domain binding human serum albumin (HSA). FIG. 1 depicts one non-limiting example of a trispecific antigen-binding protein.

In one aspect, the trispecific antigen-binding proteins comprise a domain (A) which specifically binds to innate immune cells, a domain (B) which specifically binds to human serum albumin (HSA), and a domain (C) which specifically binds to a target antigen. The three domains in trispecific antigen-binding proteins are arranged in any order. Thus, it is contemplated that the domain order of the trispecific antigen-binding proteins are:

H₂N-(A)-(B)-(C)-COOH,

H₂N-(A)-(C)-(B)-COOH,

H₂N-(B)-(A)-(C)-COOH,

H₂N-(B)-(C)-(A)-COOH,

H₂N-(C)-(B)-(A)-COOH, or

H₂N-(C)-(A)-(B)-COOH.

In some embodiments, the trispecific antigen-binding proteins have a domain order of H₂N-(A)-(B)-(C)-COOH. In some embodiments, the trispecific antigen-binding proteins have a domain order of H₂N-(A)-(C)-(B)-COOH. In some embodiments, the trispecific antigen-binding proteins have a domain order of H₂N-(B)-(A)-(C)-COOH. In some embodiments, the trispecific antigen-binding proteins have a domain order of H₂N-(B)-(C)-(A)-COOH. In some embodiments, the trispecific antigen-binding proteins have a domain order of H₂N-(C)-(B)-(A)-COOH. In some embodiments, the trispecific antigen-binding proteins have a domain order of H₂N-(C)-(A)-(B)-COOH.

Trispecific antigen-binding proteins described herein optionally comprise a polypeptide having a sequence described in Table 1 (SEQ ID NOS: 1-40) and subsequences thereof. In some embodiments, the trispecific antigen binding protein comprises a polypeptide having at least 70%-95% or more homology to a sequence described in Table 1 (SEQ ID NOS: 1-40). In some embodiments, the trispecific antigen binding protein comprises a polypeptide having at least 70%, 75%, 80%, 85%, 90%, 95%, or more homology to a sequence described in Table 1 (SEQ ID NO: 1-40). In some embodiments, the trispecific antigen binding protein has a sequence comprising at least a portion of a sequence described in Table 1 (SEQ ID NOS: 1-40). In some embodiments, the trispecific antigen-binding protein comprises a polypeptide comprising one or more of the sequences described in Table 1 (SEQ ID NOS: 1-40).

The trispecific antigen-binding proteins described herein are designed to allow specific targeting of cells expressing a target antigen by recruiting innate immune cells. This improves efficacy compared to ADCC (antibody dependent cell-mediated cytotoxicity), which is using full length antibodies directed to a sole antigen and is not capable of directly recruiting innate immune cells. In contrast, by engaging cell surface markers molecules expressed specifically on these cells, the trispecific antigen-binding proteins can crosslink cytotoxic innate immune cells with cells expressing a target antigen in a highly specific fashion, thereby directing the immune response of the innate immune cell towards the target cell. The trispecific antigen-binding proteins described herein engage innate immune cells via binding to the surface-expressed proteins. Simultaneous binding of several trispecific antigen-binding protein to an innate immune cell and to a target antigen expressed on the surface of particular cells causes innate immune cell activation and mediates the subsequent lysis of the particular target antigen expressing cell. Thus, trispecific antigen-binding proteins are contemplated to display strong, specific and efficient target cell killing. In some embodiments, the trispecific antigen-binding proteins described herein stimulate target cell killing by innate immune cells to eliminate pathogenic cells (e.g., tumor cells, virally or bacterially infected cells, autoreactive T cells, etc). In some of such embodiments, cells are eliminated selectively, thereby reducing the potential for toxic side effects. In other embodiments, the same polypeptides could be used to enhance the elimination of endogenous cells for therapeutic effect, such as B or T lymphocytes in autoimmune disease, or hematopoietic stem cells (HSCs) for stem cell transplantation.

The trispecific antigen-binding proteins described herein confer further therapeutic advantages over traditional monoclonal antibodies and other smaller bispecific molecules. Generally, the effectiveness of recombinant protein pharmaceuticals depends heavily on the intrinsic pharmacokinetics of the protein itself. One such benefit here is that the trispecific antigen-binding proteins described herein have extended pharmacokinetic elimination half-time due to having a half-life extension domain such as a domain specific to HSA. In this respect, the trispecific antigen-binding proteins described herein have an extended serum elimination half-time of about two, three, about five, about seven, about 10, about 12, or about 14 days in some embodiments. This contrasts to other binding proteins such as BiTE or DART molecules which have relatively much shorter elimination half-times. For example, the BiTE CD19×CD3 bispecific scFv-scFv fusion molecule requires continuous intravenous infusion (i.v.) drug delivery due to its short elimination half-time. The longer intrinsic half-times of the trispecific antigen-binding proteins solve this issue thereby allowing for increased therapeutic potential such as low-dose pharmaceutical formulations, decreased periodic administration and/or novel pharmaceutical compositions.

The trispecific antigen-binding proteins described herein also have an optimal size for enhanced tissue penetration and tissue distribution. Larger sizes limit or prevent penetration or distribution of the protein in the target tissues. The trispecific antigen-binding proteins described herein avoid this by having a small size that allows enhanced tissue penetration and distribution. Accordingly, the trispecific antigen-binding proteins described herein, in some embodiments have a size of about 50 kD to about 80 kD, about 50 kD to about 75 kD, about 50 kD to about 70 kD, or about 50 kD to about 65 kD. Thus, the size of the trispecific antigen-binding proteins is advantageous over IgG antibodies which are about 150 kD and the BiTE and DART diabody molecules which are about 55 kD but are not half-life extended and therefore cleared quickly through the kidney.

In further embodiments, the trispecific antigen-binding proteins described herein have an optimal size for enhanced tissue penetration and distribution. In these embodiments, the trispecific antigen-binding proteins are constructed to be as small as possible, while retaining specificity toward its targets. Accordingly, in these embodiments, the trispecific antigen-binding proteins described herein have a size of about 20 kD to about 40 kD or about 25 kD to about 35 kD to about 40 kD, to about 45 kD, to about 50 kD, to about 55 kD, to about 60 kD, to about 65 kD. In some embodiments, the trispecific antigen-binding proteins described herein have a size of about 50 kD, 49, kD, 48 kD, 47 kD, 46 kD, 45 kD, 44 kD, 43 kD, 42 kD, 41 kD, 40 kD, about 39 kD, about 38 kD, about 37 kD, about 36 kD, about 35 kD, about 34 kD, about 33 kD, about 32 kD, about 31 kD, about 30 kD, about 29 kD, about 28 kD, about 27 kD, about 26 kD, about 25 kD, about 24 kD, about 23 kD, about 22 kD, about 21 kD, or about 20 kD. An exemplary approach to the small size is through the use of single domain antibody (sdAb) fragments for each of the domains. For example, a particular trispecific antigen-binding protein has an anti-innate immune cell sdAb, anti-HSA sdAb and an sdAb for a target antigen. This reduces the size of the exemplary trispecific antigen-binding protein to under 40 kD. Thus in some embodiments, the domains of the trispecific antigen-binding proteins are all single domain antibody (sdAb) fragments. In other embodiments, the trispecific antigen-binding proteins described herein comprise small molecule entity (SME) binders for HSA and/or the target antigen. SME binders are small molecules averaging about 500 to 2000 Da in size and are attached to the trispecific antigen-binding proteins by known methods, such as sortase ligation or conjugation. In these instances, one of the domains of a trispecific antigen-binding protein is a sortase recognition sequence, e.g., LPETG (SEQ ID NO: 48). To attach a SME binder to a trispecific antigen-binding protein with a sortase recognition sequence, the protein is incubated with a sortase and a SME binder whereby the sortase attaches the SME binder to the recognition sequence. Known SME binders include MIP-1072 and MIP-1095 which bind to prostate-specific membrane antigen (PSMA). In yet other embodiments, the domain which binds to a target antigen of a trispecific antigen-binding proteins described herein comprise a knottin peptide for binding a target antigen. Knottins are disufide-stabilized peptides with a cysteine knot scaffold and have average sizes about 3.5 kD. Knottins have been contemplated for binding to certain tumor molecules such as fibronectin and VEGF-receptor. In further embodiments, domain which binds to a target antigen of a trispecific antigen-binding proteins described herein comprise a natural receptor ligand such as B-cell activating factor (BAFF/BLyS).

Another feature of the trispecific antigen-binding proteins described herein is that they are of a single-polypeptide design with flexible linkage of their domains. This allows for facile production and manufacturing of the trispecific antigen-binding proteins as they can be encoded by single cDNA molecule to be easily incorporated into a vector. Further, because the trispecific antigen-binding proteins described herein are a monomeric single polypeptide chain, there are no chain pairing issues or a requirement for dimerization. It is contemplated that the trispecific antigen-binding proteins described herein have a reduced tendency to aggregate unlike other reported molecules such as bispecific proteins with Fc-gamma immunoglobulin domains.

In the trispecific antigen-binding proteins described herein, the domains are linked by internal linkers L1 and L2, where L1 links the first and second domain of the trispecific antigen-binding proteins and L2 links the second and third domains of the trispecific antigen-binding proteins. Linkers L1 and L2 have an optimized length and/or amino acid composition. In some embodiments, linkers L1 and L2 are the same length and amino acid composition. In other embodiments, L1 and L2 are different. In certain embodiments, internal linkers L1 and/or L2 are “short”, i.e., consist of 0, 1, 2, 3, 4, 5, 6, 7, 8, 9, 10, 11 or 12 amino acid residues. Thus, in certain instances, the internal linkers consist of about 12 or less amino acid residues. In the case of 0 amino acid residues, the internal linker is a peptide bond. In certain embodiments, internal linkers L1 and/or L2 are “long”, i.e., consist of 15, 20 or 25 amino acid residues. In some embodiments, these internal linkers consist of about 3 to about 15, for example 8, 9 or 10 contiguous amino acid residues. Regarding the amino acid composition of the internal linkers L1 and L2, peptides are selected with properties that confer flexibility to the trispecific antigen-binding proteins, do not interfere with the binding domains as well as resist cleavage from proteases. For example, glycine and serine residues generally provide protease resistance. Examples of internal linkers suitable for linking the domains in the trispecific antigen-binding proteins include but are not limited to (GS)ₙ (SEQ ID NO: 41), (GGS)ₙ (SEQ ID NO: 42), (GGGS)ₙ (SEQ ID NO: 43), (GGSG)ₙ (SEQ ID NO: 44), (GGSGG)ₙ (SEQ ID NO: 45), or (GGGGS)ₙ (SEQ ID NO: 46), wherein n is 1, 2, 3, 4, 5, 6, 7, 8, 9, or 10. In one embodiment, internal linker L1 and/or L2 is (GGGGS)₄ (SEQ ID NO: 47) or (GGGGS)₃ (SEQ ID NO: 48).

### Innate Immune Cell Binding Domain

Innate immune cells are a part of the innate immune system. Innate immune cells recognize and respond to pathogens via recognition of pathogens. They play a variety of roles in immunity including but not limited to, recruitment of other immune cells via cytokine secretion, activation of the complement cascade, identification and removal of foreign substances in the body, and presenting antigen to other immune cells. Innate immune cells include multiple cell types such as dendritic cells, plasmacytoid dendritic cells, natural killer cells, natural killer T cells, monocytes, neutrophils, basophils, and eosinophils. In some cases, innate immune cells are not T cells. Recruitment and activation of innate immune cells to the site of a target antigen, for example at a diseased cell or tissue leads to activation of the immune system at that site. Each innate immune cell, is recruited by at least one cell surface marker expressed by the cell, which is specific for the innate immune cell type. Non-limiting examples of innate immune cell surface markers include but are not limited to in dendritic cells: CD1c, CD32, CD83, CD141, CD209, and WIC II; plasmacytoid dendritic cells: CD123, CD303, and CD304; natural killer cells: CD16, CD56; natural killer T cells: CD1d, CD160, PLZF, NKG2D, and CD94-NKG2A/C/E; monocytes: CD14, CD16, CD47, and CD64; neutrophils: CD15, CD16, and CD89; basophils: 2D7 antigen, CD123, CD203c, and FcεRIα; eosinophils: CD11b, CD193, EMR1, and Siglec-8. In one aspect, the trispecific antigen-binding proteins described herein comprise a domain which specifically binds to an innate immune cell. In one aspect, the trispecific antigen-binding proteins described herein comprise a domain which specifically binds to a human innate immune cell. In some embodiments, the tri-specific antigen-binding proteins described herein comprise a domain that specifically binds to an innate immune cell surface marker including but are not limited to CD1c, CD83, CD141, CD209, WIC II, CD123, CD303, CD304, CD16, CD56, CD1d, CD160, PLZF, NKG2d, CD94-NKG2A/C/E, CD14, CD16, CD47, CD64, CD15 CD16, CD89, 2D7 antigen, CD123, CD203c, FcεRIα, CD11b, CD193, EMR1, and Siglec-8. In some embodiments, the trispecific antigen-binding proteins described herein comprise a domain which specifically binds to a dendritic cell. In some embodiments, the trispecific antigen-binding proteins described herein comprise a domain which specifically binds to a plasmacytoid dendritic cell. In some embodiments, the trispecific antigen-binding proteins described herein comprise a domain which specifically binds to a natural killer cell. In some embodiments, the antigen-binding proteins described herein comprise two or more domains which when activated by a protease specifically bind to a natural killer T cell. In some embodiments, the antigen-binding proteins described herein comprise two or more domains which when activated by a protease specifically bind to a monocyte. In some embodiments, the antigen-binding proteins described herein comprise two or more domains which when activated by a protease specifically bind to a neutrophil. In some embodiments, the antigen-binding proteins described herein comprise two or more domains which when activated by a protease specifically bind to a eosinophil. In some embodiments, the antigen-binding proteins described herein comprise two or more domains which when activated by a protease specifically bind to a basophil.

In certain embodiments, the innate immune cell binding domain of the trispecific antigen-binding proteins described herein exhibit not only potent innate immune cell binding affinities with a human innate immune cell surface marker, but show also excellent crossreactivity with the respective a cynomolgus monkey innate immune cell surface proteins. In some instances, the innate immune cell binding domain of the trispecific antigen-binding proteins are cross-reactive with an innate immune cell from cynomolgus monkey. In certain instances, human:cynomolgous K_{D} ratios for an innate cell surface marker are between 5 and 0.2.

In some embodiments, the innate immune cell binding domain of the trispecific antigen-binding protein can be any domain that binds to an innate immune cell including but not limited to domains from a monoclonal antibody, a polyclonal antibody, a recombinant antibody, a human antibody, a humanized antibody. In some instances, it is beneficial for the innate immune cell binding domain to be derived from the same species in which the trispecific antigen-binding protein will ultimately be used in. For example, for use in humans, it may be beneficial for the innate immune cell binding domain of the trispecific antigen-binding protein to comprise human or humanized residues from the antigen binding domain of an antibody or antibody fragment.

Thus, in one aspect, the antigen-binding domain comprises a humanized or human antibody or an antibody fragment, or a murine antibody or antibody fragment. In one embodiment, the humanized or human anti-innate immune cell binding domain comprises one or more (e.g., all three) light chain complementary determining region 1 (LC CDR1), light chain complementary determining region 2 (LC CDR2), and light chain complementary determining region 3 (LC CDR3) of a humanized or human anti-innate immune cell binding domain described herein, and/or one or more (e.g., all three) heavy chain complementary determining region 1 (HC CDR1), heavy chain complementary determining region 2 (HC CDR2), and heavy chain complementary determining region 3 (HC CDR3) of a humanized or human anti-innate immune cell binding domain described herein, e.g., a humanized or human anti-innate immune cell binding domain comprising one or more, e.g., all three, LC CDRs and one or more, e.g., all three, HC CDRs.

In some embodiments, the humanized or human anti-innate immune cell binding domain comprises a humanized or human light chain variable region specific to an innate immune cell surface marker where the light chain variable region specific to an innate immune cell surface marker comprises human or non-human light chain CDRs in a human light chain framework region. In certain instances, the light chain framework region is a λ (lamda) light chain framework. In other instances, the light chain framework region is a κ (kappa) light chain framework.

In some embodiments, the humanized or human anti-innate immune cell binding domain comprises a humanized or human heavy chain variable region specific to an innate immune cell surface marker where the heavy chain variable region specific to innate immune cell comprises human or non-human heavy chain CDRs in a human heavy chain framework region.

In certain instances, the complementary determining regions of the heavy chain and/or the light chain are derived from known anti-innate immune cell antibodies, such as, for example, Lorvotuzumab, 3C12C, CSL362, 3G8, rMil2, E4, NNC141-0100.

In one embodiment, the anti-innate immune cell binding domain is a single chain variable fragment (scFv) comprising a light chain and a heavy chain of an amino acid sequence provided herein. As used herein, “single chain variable fragment” or “scFv” refers to an antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single polypeptide chain, and wherein the scFv retains the specificity of the intact antibody from which it is derived. In an embodiment, the anti-innate immune cell binding domain comprises: a light chain variable region comprising an amino acid sequence having at least one, two or three modifications (e.g., substitutions) but not more than 30, 20 or 10 modifications (e.g., substitutions) of an amino acid sequence of a light chain variable region provided herein, or a sequence with 95-99% identity with an amino acid sequence provided herein; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided herein, or a sequence with 95-99% identity to an amino acid sequence provided herein. In one embodiment, the humanized or human anti-innate immune cell binding domain is a scFv, and a light chain variable region comprising an amino acid sequence described herein, is attached to a heavy chain variable region comprising an amino acid sequence described herein, via a scFv linker. The light chain variable region and heavy chain variable region of a scFv can be, e.g., in any of the following orientations: light chain variable region-scFv linker-heavy chain variable region or heavy chain variable region-scFv linker-light chain variable region.

In some instances, scFvs which bind to innate immune cell are prepared according to known methods. For example, scFv molecules can be produced by linking VH and VL regions together using flexible polypeptide linkers. The scFv molecules comprise a scFv linker (e.g., a Ser-Gly linker) with an optimized length and/or amino acid composition. Accordingly, in some embodiments, the length of the scFv linker is such that the VH or VL domain can associate intermolecularly with the other variable domain to form the innate immune cell binding site. In certain embodiments, such scFv linkers are “short”, i.e. consist of 0, 1, 2, 3, 4, 5, 6, 7, 8, 9, 10, 11 or 12 amino acid residues. Thus, in certain instances, the scFv linkers consist of about 12 or less amino acid residues. In the case of 0 amino acid residues, the scFv linker is a peptide bond. In some embodiments, these scFv linkers consist of about 3 to about 15, for example 8, 9 or 10 contiguous amino acid residues. Regarding the amino acid composition of the scFv linkers, peptides are selected that confer flexibility, do not interfere with the variable domains as well as allow inter-chain folding to bring the two variable domains together to form a functional innate immune cell binding site. For example, scFv linkers comprising glycine and serine residues generally provide protease resistance. In some embodiments, linkers in a scFv comprise glycine and serine residues. The amino acid sequence of the scFv linkers can be optimized, for example, by phage-display methods to improve the innate immune cell binding and production yield of the scFv. Examples of peptide scFv linkers suitable for linking a variable light chain domain and a variable heavy chain domain in a scFv include but are not limited to (GS)ₙ (SEQ ID NO: 41), (GGS)ₙ (SEQ ID NO: 42), (GGGS)ₙ (SEQ ID NO: 43), (GGSG)ₙ (SEQ ID NO: 44), (GGSGG)ₙ (SEQ ID NO: 45), or (GGGGS)ₙ (SEQ ID NO: 46), wherein n is 1, 2, 3, 4, 5, 6, 7, 8, 9, or 10. In one embodiment, the scFv linker can be (GGGGS)₄ (SEQ ID NO: 55) or (GGGGS)₃ (SEQ ID NO: 47). Variation in the linker length may retain or enhance activity, giving rise to superior efficacy in activity studies.

In some embodiments, innate immune cell binding domain of a trispecific antigen-binding protein has an affinity to an innate immune cell surface marker on innate immune cells with a K_{D} of 1000 nM or less, 500 nM or less, 200 nM or less, 100 nM or less, 80 nM or less, 50 nM or less, 20 nM or less, 10 nM or less, 5 nM or less, 1 nM or less, or 0.5 nM or less. In some embodiments, the innate immune cell binding domain of a trispecific antigen-binding protein has an affinity to an innate immune cell surface marker with a K_{D} of 1000 nM or less, 500 nM or less, 200 nM or less, 100 nM or less, 80 nM or less, 50 nM or less, 20 nM or less, 10 nM or less, 5 nM or less, 1 nM or less, or 0.5 nM or less. In further embodiments, innate immune cell binding domain of a trispecific antigen-binding protein has low affinity to innate immune cell, i.e., about 100 nM or greater.

The affinity to bind to innate immune cell can be determined, for example, by the ability of the trispecific antigen-binding protein itself or its innate immune cell binding domain to bind to innate immune cell coated on an assay plate; displayed on a microbial cell surface; in solution; etc. The binding activity of the trispecific antigen-binding protein itself or its innate immune cell binding domain of the present disclosure to innate immune cell can be assayed by immobilizing the ligand (e.g., an innate immune cell surface marker) or the trispecific antigen-binding protein itself or its innate immune cell binding domain, to a bead, substrate, cell, etc. Agents can be added in an appropriate buffer and the binding partners incubated for a period of time at a given temperature. After washes to remove unbound material, the bound protein can be released with, for example, SDS, buffers with a high pH, and the like and analyzed, for example, by Surface Plasmon Resonance (SPR).

### Half-Life Extension Domain

Contemplated herein are domains which extend the half-life of an antigen-binding domain. Such domains are contemplated to include but are not limited to HSA binding domains, Fc domains, small molecules, and other half-life extension domains known in the art. In some embodiments, Fc and albumin binding domains extend half-lives by increasing the size of the peptide drug. In some embodiments, Fc and albumin binding domains bind to the neonatal Fc receptor, FcRn, which, at least in some cases prevents degradation of the fusion protein in the endosome. In some embodiments, Fc domains, or HSA binding domains improve solubility and stability of the antigen binding domain.

Human serum albumin (HSA) (molecular mass ˜67 kDa) is the most abundant protein in plasma, present at about 50 mg/ml (600 μM), and has a half-life of around 20 days in humans. HSA serves to maintain plasma pH, contributes to colloidal blood pressure, functions as carrier of many metabolites and fatty acids, and serves as a major drug transport protein in plasma.

Noncovalent association with albumin extends the elimination half-time of short lived proteins. For example, a recombinant fusion of an albumin binding domain to a Fab fragment resulted in an in vivo clearance of 25- and 58-fold and a half-life extension of 26- and 37-fold when administered intravenously to mice and rabbits respectively as compared to the administration of the Fab fragment alone. In another example, when insulin is acylated with fatty acids to promote association with albumin, a protracted effect was observed when injected subcutaneously in rabbits or pigs. Together, these studies demonstrate a linkage between albumin binding and prolonged action.

In one aspect, the trispecific antigen-binding proteins described herein comprise a half-life extension domain, for example a domain which specifically binds to HSA. In some embodiments, the HSA binding domain of a trispecific antigen-binding protein can be any domain that binds to HSA including but not limited to domains from a monoclonal antibody, a polyclonal antibody, a recombinant antibody, a human antibody, a humanized antibody. In some embodiments, the HSA binding domain is a single chain variable fragments (scFv), single-domain antibody such as a heavy chain variable domain (VH), a light chain variable domain (VL) and a variable domain (VHH) of camelid derived single domain antibody, peptide, ligand or small molecule entity specific for HSA. In certain embodiments, the HSA binding domain is a single-domain antibody. In other embodiments, the HSA binding domain is a peptide. In further embodiments, the HSA binding domain is a small molecule. It is contemplated that the HSA binding domain of a trispecific antigen-binding protein is fairly small and no more than 25 kD, no more than 20 kD, no more than 15 kD, or no more than 10 kD in some embodiments. In certain instances, the HSA binding is 5 kD or less if it is a peptide or small molecule entity.

The half-life extension domain of a trispecific antigen-binding protein provides for altered pharmacodynamics and pharmacokinetics of the trispecific antigen-binding protein itself. As above, the half-life extension domain extends the elimination half-time. The half-life extension domain also alters pharmacodynamic properties including alteration of tissue distribution, penetration, and diffusion of the trispecific antigen-binding protein. In some embodiments, the half-life extension domain provides for improved tissue (including tumor) targeting, tissue distribution, tissue penetration, diffusion within the tissue, and enhanced efficacy as compared with a protein without an half-life extension domain. In one embodiment, therapeutic methods effectively and efficiently utilize a reduced amount of the trispecific antigen-binding protein, resulting in reduced side effects, such as reduced non-tumor cell cytotoxicity.

Further, the binding affinity of the half-life extension domain can be selected so as to target a specific elimination half-time in a particular trispecific antigen-binding protein. Thus, in some embodiments, the half-life extension domain has a high binding affinity. In other embodiments, the half-life extension domain has a medium binding affinity. In yet other embodiments, the half-life extension domain has a low or marginal binding affinity. Exemplary binding affinities include K_{D} concentrations at 10 nM or less (high), between 10 nM and 100 nM (medium), and greater than 100 nM (low). As above, binding affinities to HSA are determined by known methods such as Surface Plasmon Resonance (SPR).

The half-life extension domain of a trispecific antigen binding protein extends the half-life of a trispecific antigen binding protein to at least about two days, about three days, about four days, about five days, about six days, about seven days, about eight days, about nine days, about ten days, or more. In some embodiments, the half-life extension domain extends the half-life of a trispecific antigen binding protein to at least 2-3 days, 3-4 days, 4-5, days, 5-6 days, 6-7 days, 7-8 days, 2-5 days, 5-8 days, 2-10 days, or more. In some embodiments, the half-life extension domain extends the half-life of a trispecific antigen binding domain to at least about five days. In some embodiments, the half-life extension domain extends the half-life of a trispecific antigen binding protein to five or more days.

### Target Antigen Binding Domain

In addition to the described innate immune cell binding and half-life extension domains, the trispecific antigen-binding proteins described herein also comprise a domain that binds to a target antigen. A target antigen is involved in and/or associated with a disease, disorder or condition. In particular, a target antigen associated with a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease. In some embodiments, a target antigen is a tumor antigen expressed on a tumor cell. Alternatively in some embodiments, a target antigen is associated with a pathogen such as a virus or bacterium.

In some embodiments, a target antigen is a cell surface molecule such as a protein, lipid or polysaccharide. In some embodiments, a target antigen is a on a tumor cell, virally infected cell, bacterially infected cell, damaged red blood cell, arterial plaque cell, or fibrotic tissue cell. Target antigens, in some cases, are expressed on the surface of a diseased cell or tissue, for example a tumor or a cancer cell. Target antigens include but are not limited to EpCAM, EGFR, HER-2, HER-3, c-Met, FoIR, PSMA, MSLN, and CEA.

The design of the trispecific antigen-binding proteins described herein allows the binding domain to a target antigen to be flexible in that the binding domain to a target antigen can be any type of binding domain, including but not limited to, domains from a monoclonal antibody, a polyclonal antibody, a recombinant antibody, a human antibody, a humanized antibody. In some embodiments, the binding domain to a target antigen is a single chain variable fragments (scFv), single-domain antibody such as a heavy chain variable domain (VH), a light chain variable domain (VL) and a variable domain (VHH) of camelid derived single domain antibody. In other embodiments, the binding domain to a target antigen is a non-Ig binding domain, i.e., antibody mimetic, such as anticalins, affilins, affibody molecules, affimers, affitins, alphabodies, avimers, DARPins, fynomers, kunitz domain peptides, and monobodies. In further embodiments, the binding domain to a target antigen is a ligand or peptide that binds to or associates with a target antigen. In yet further embodiments, the binding domain to a target antigen is a knottin. In yet further embodiments, the binding domain to a target antigen is a small molecular entity.

### Trispecific Protein Modifications

The trispecific antigen-binding proteins described herein encompass derivatives or analogs in which (i) an amino acid is substituted with an amino acid residue that is not one encoded by the genetic code, (ii) the mature polypeptide is fused with another compound such as polyethylene glycol, or (iii) additional amino acids are fused to the protein, such as a leader or secretory sequence or a sequence for purification of the protein.

Typical modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Modifications are made anywhere in trispecific antigen-binding proteins described herein, including the peptide backbone, the amino acid side-chains, and the amino or carboxyl termini. Certain common peptide modifications that are useful for modification of trispecific antigen-binding proteins include glycosylation, lipid attachment, sulfation, gamma-carboxylation of glutamic acid residues, hydroxylation, blockage of the amino or carboxyl group in a polypeptide, or both, by a covalent modification, and ADP-ribosylation.

### Polynucleotides Encoding Trispecific Antigen-Binding Proteins

Also provided, in some embodiments, are polynucleotide molecules encoding a trispecific antigen-binding protein described herein. In some embodiments, the polynucleotide molecules are provided as a DNA construct. In other embodiments, the polynucleotide molecules are provided as a messenger RNA transcript.

The polynucleotide molecules are constructed by known methods such as by combining the genes encoding the three binding domains either separated by peptide linkers or, in other embodiments, directly linked by a peptide bond, into a single genetic construct operably linked to a suitable promoter, and optionally a suitable transcription terminator, and expressing it in bacteria or other appropriate expression system such as, for example CHO cells. In the embodiments where the target antigen binding domain is a small molecule, the polynucleotides contain genes encoding the innate immune cell binding domain and the half-life extension domain. In the embodiments where the half-life extension domain is a small molecule, the polynucleotides contain genes encoding the domains that bind to the innate immune cell and the target antigen. Depending on the vector system and host utilized, any number of suitable transcription and translation elements, including constitutive and inducible promoters, may be used. The promoter is selected such that it drives the expression of the polynucleotide in the respective host cell.

In some embodiments, the polynucleotide is inserted into a vector, preferably an expression vector, which represents a further embodiment. This recombinant vector can be constructed according to known methods. Vectors of particular interest include plasmids, phagemids, phage derivatives, virii (e.g., retroviruses, adenoviruses, adeno-associated viruses, herpes viruses, lentiviruses, and the like), and cosmids.

A variety of expression vector/host systems may be utilized to contain and express the polynucleotide encoding the polypeptide of the described trispecific antigen-binding protein. Examples of expression vectors for expression in E. coli are pSKK (Le Gall et al., J Immunol Methods. (2004) 285(1):111-27) or pcDNA5 (Invitrogen) for expression in mammalian cells.

Thus, the trispecific antigen-binding proteins as described herein, in some embodiments, are produced by introducing a vector encoding the protein as described above into a host cell and culturing said host cell under conditions whereby the protein domains are expressed, may be isolated and, optionally, further purified.

### Pharmaceutical Compositions

Also provided, in some embodiments, are pharmaceutical compositions comprising a trispecific antigen-binding protein described herein, a vector comprising the polynucleotide encoding the polypeptide of the trispecific antigen-binding proteins or a host cell transformed by this vector and at least one pharmaceutically acceptable carrier. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents.

In some embodiments of the pharmaceutical compositions, the trispecific antigen-binding protein described herein is encapsulated in nanoparticles. In some embodiments, the nanoparticles are fullerenes, liquid crystals, liposome, quantum dots, superparamagnetic nanoparticles, dendrimers, or nanorods. In other embodiments of the pharmaceutical compositions, the trispecific antigen-binding protein is attached to liposomes. In some instances, the trispecific antigen-binding protein are conjugated to the surface of liposomes. In some instances, the trispecific antigen-binding protein are encapsulated within the shell of a liposome. In some instances, the liposome is a cationic liposome.

The trispecific antigen-binding proteins described herein are contemplated for use as a medicament. Administration is effected by different ways, e.g. by intravenous, intraperitoneal, subcutaneous, intramuscular, topical or intradermal administration. In some embodiments, the route of administration depends on the kind of therapy and the kind of compound contained in the pharmaceutical composition. The dosage regimen will be determined by the attending physician and other clinical factors. Dosages for any one patient depends on many factors, including the patient's size, body surface area, age, sex, the particular compound to be administered, time and route of administration, the kind of therapy, general health and other drugs being administered concurrently. An “effective dose” refers to amounts of the active ingredient that are sufficient to affect the course and the severity of the disease, leading to the reduction or remission of such pathology and may be determined using known methods.

### Methods of Treatment

Also provided herein, in some embodiments, are methods and uses for stimulating the immune system of an individual in need thereof comprising administration of a trispecific antigen-binding protein described herein. In some instances, the administration of a trispecific antigen-binding protein described herein induces and/or sustains cytotoxicity towards a cell expressing a target antigen. In some instances, the cell expressing a target antigen is a cancer or tumor cell, a virally infected cell, a bacterially infected cell, an autoreactive T or B cell, damaged red blood cells, arterial plaques, or fibrotic tissue.

In some cases, the administration of a trispecific antigen-binding protein described herein induces an innate immune response against cancer cells, by activation of the innate immune system of an individual. In some cases, recruitment of an innate immune cell to a tumor micro-environment, by the trispecific antigen-binding protein of the present disclosure, results in induction the innate immune system, leading to cytotoxic or an anti-tumor response in a tumor microenvironment. As used herein, the term “innate immune response,” or “induction of innate immune system,” refers to non-antigen-specific responses in cells and tissues in a human or animal, preferably differentially or preferentially induced in tumor cells. It also refers to cellular changes that affect the abilities of cells to act as antigen-presenting cells and/or modulate the antigen-specific adaptive immune response. In some embodiments, the innate immune response comprises activation of natural killer (NK) cell activity. In some embodiments, the innate immune response comprises production and/or secretion of one or more cytokines or growth factors such as in non-limiting examples IFN-α, IFN-γ, IL-1, IL-6, IL-10, IL-12, and TNF-α. Innate immunity further comprises, in some cases, involvement of macrophages, dendritic cells and monocytes. Also provided herein are methods of monitoring the induction of innate immune response, upon administration of a trispecific antigen-binding protein of the present disclosure, by evaluating the expression levels of innate immune cell activation markers, e.g., CD56 (NK cell activation marker), CD69 (NK cell activation marker), CD14 (monocyte activation marker), CD86 (B cell activation marker), CD80 (B7-1 activation marker). In some embodiments, high expression of an innate immune cell activation marker is correlated with induction of innate immune response and enhanced cytotoxic or anti-tumor response of the trispecific antigen-binding protein, towards a cell expressing a target antigen.

Also provided herein are methods and uses for a treatment of a disease, disorder or condition associated with a target antigen comprising administering to an individual in need thereof a trispecific antigen-binding protein described herein. Diseases, disorders or conditions associated with a target antigen include, but are not limited to, viral infection, bacterial infection, auto-immune disease, transplant rejection, atherosclerosis, or fibrosis. In other embodiments, the disease, disorder or condition associated with a target antigen is a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease. In one embodiment, the disease, disorder or condition associated with a target antigen is cancer. In one instance, the cancer is a hematological cancer. In another instance, the cancer is a solid tumor cancer.

As used herein, in some embodiments, “treatment” or “treating” or “treated” refers to therapeutic treatment wherein the object is to slow (lessen) an undesired physiological condition, disorder or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. In other embodiments, “treatment” or “treating” or “treated” refers to prophylactic measures, wherein the object is to delay onset of or reduce severity of an undesired physiological condition, disorder or disease, such as, for example is a person who is predisposed to a disease (e.g., an individual who carries a genetic marker for a disease such as breast cancer).

In some embodiments of the methods described herein, the trispecific antigen-binding proteins are administered in combination with an agent for treatment of the particular disease, disorder or condition. Agents include but are not limited to, therapies involving antibodies, small molecules (e.g., chemotherapeutics), hormones (steroidal, peptide, and the like), radiotherapies (γ-rays, X-rays, and/or the directed delivery of radioisotopes, microwaves, UV radiation and the like), gene therapies (e.g., antisense, retroviral therapy and the like) and other immunotherapies. In some embodiments, the trispecific antigen-binding proteins are administered in combination with anti-diarrheal agents, anti-emetic agents, analgesics, opioids and/or non-steroidal anti-inflamatory agents. In some embodiments, the trispecific antigen-binding proteins are administered before, during, or after surgery.

### Certain Definitions

As used herein, “elimination half-time” is used in its ordinary sense, as is described in Goodman and Gillman's The Pharmaceutical Basis of Therapeutics 21-25 (Alfred Goodman Gilman, Louis S. Goodman, and Alfred Gilman, eds., 6th ed. 1980). Briefly, the term is meant to encompass a quantitative measure of the time course of drug elimination. The elimination of most drugs is exponential (i.e., follows first-order kinetics), since drug concentrations usually do not approach those required for saturation of the elimination process. The rate of an exponential process may be expressed by its rate constant, k, which expresses the fractional change per unit of time, or by its half-time, t_{1/2} the time required for 50% completion of the process. The units of these two constants are time⁻¹ and time, respectively. A first-order rate constant and the half-time of the reaction are simply related (k×t_{1/2}=0.693) and may be interchanged accordingly. Since first-order elimination kinetics dictates that a constant fraction of drug is lost per unit time, a plot of the log of drug concentration versus time is linear at all times following the initial distribution phase (i.e. after drug absorption and distribution are complete). The half-time for drug elimination can be accurately determined from such a graph.

### EXAMPLES

### Example 1: Construction of an Exemplary Trispecific Antigen-Binding Protein to CD20

Generation of a scFv CD16 Binding Domain

The human CD16 canonical sequence is Uniprot Accession No. P08637. Antibodies against CD16 are generated via known technologies such as affinity maturation. Where murine anti-CD16 antibodies are used as a starting material, humanization of murine anti-CD16 antibodies is desired for the clinical setting, where the mouse-specific residues may induce a human-anti-mouse antigen (HAMA) response in subjects who receive treatment of a trispecific antigen-binding protein described herein. Humanization is accomplished by grafting CDR regions from murine anti-CD16 antibody onto appropriate human germline acceptor frameworks, optionally including other modifications to CDR and/or framework regions. As provided herein, antibody and antibody fragment residue numbering follows Kabat (Kabat E. A. et al, 1991; Chothia et al, 1987).

Human or humanized anti-CD16 antibodies are therefore used to generate scFv sequences for CD16 binding domains of a trispecific antigen-binding protein. DNA sequences coding for human or humanized VL and VH domains are obtained, and the codons for the constructs are, optionally, optimized for expression in cells from Homo sapiens. The order in which the VL and VH domains appear in the scFv is varied (i.e., VL-VH, or VH-VL orientation), and three copies of the “G4S” (SEQ ID NO: 49) or “G₄S” (SEQ ID NO: 49) subunit (G₄S)₃ (SEQ ID NO: 48) connect the variable domains to create the scFv domain. Anti-CD16 scFv plasmid constructs can have optional Flag, His or other affinity tags, and are electroporated into HEK293 or other suitable human or mammalian cell lines and purified. Validation assays include binding analysis by FACS, kinetic analysis using Proteon, and staining of CD16-expressing cells.

Generation of a scFv CD20 Binding Domain

CD20 is one of the cell surface proteins present on B-lymphocytes. CD20 antigen is found in normal and malignant pre-B and mature B lymphocytes, including those in over 90% of B-cell non-Hodgkin's lymphomas (NHL). The antigen is absent in hematopoetic stem cells, activated B lymphocytes (plasma cells) and normal tissue. As such, several antibodies mostly of murine origin have been described: 1F5, 2B8/C2B8, 2H7, and 1H4.

A scFv binding domain to CD20 is generated similarly to the above method for generation of a scFv binding domain to CD16.

### Cloning of DNA Expression Constructs Encoding the Trispecific Antigen-Binding Protein

The anti-CD16 scFv domains are used to construct a trispecific antigen-binding protein in combination with an anti-CD20 scFv domain and a HSA binding domain (e.g, a peptide or VH domain), with the domains organized as shown FIG. 1. For expression of a trispecific antigen-binding protein in CHO cells, coding sequences of all protein domains are cloned into a mammalian expression vector system. In brief, gene sequences encoding the CD16 binding domain, HSA binding domain, and CD20 binding domain along with peptide linkers L1 and L2 are separately synthesized and subcloned. The resulting constructs are then ligated together in the order of ‘CD20 binding domain-L1-CD16 binding domain-L2-HSA binding domain’ to yield a final construct. All expression constructs are designed to contain coding sequences for an N-terminal signal peptide and a C-terminal hexahistidine (6×His)-tag (SEQ ID NO: 50) to facilitate protein secretion and purification, respectively.

### Expression of Trispecific Antigen-Binding Proteins in Stably Transfected CHO Cells

A CHO cell expression system (Flp-In®, Life Technologies), a derivative of CHO-K1 Chinese Hamster ovary cells (ATCC, CCL-61) (Kao and Puck, Proc. Natl. Acad Sci USA 1968; 60(4):1275-81), is used. Adherent cells are subcultured according to standard cell culture protocols provided by Life Technologies.

For adaption to growth in suspension, cells are detached from tissue culture flasks and placed in serum-free medium. Suspension-adapted cells are cryopreserved in medium with 10% DMSO.

Recombinant CHO cell lines stably expressing secreted trispecific antigen-binding proteins are generated by transfection of suspension-adapted cells. During selection with the antibiotic Hygromycin B viable cell densities are measured twice a week, and cells are centrifuged and resuspended in fresh selection medium at a maximal density of 0.1×10⁶ viable cells/mL. Cell pools stably expressing trispecific antigen-binding proteins are recovered after 2-3 weeks of selection at which point cells are transferred to standard culture medium in shake flasks. Expression of recombinant secreted proteins is confirmed by performing protein gel electrophoresis or flow cytometry. Stable cell pools are cryopreserved in DMSO containing medium.

Trispecific antigen-binding proteins are produced in 10-day fed-batch cultures of stably transfected CHO cell lines by secretion into the cell culture supernatant. Cell culture supernatants are harvested after 10 days at culture viabilities of typically >75%. Samples are collected from the production cultures every other day and cell density and viability are assessed. On day of harvest, cell culture supernatants are cleared by centrifugation and vacuum filtration before further use.

Protein expression titers and product integrity in cell culture supernatants are analyzed by SDS-PAGE.

### Purification of Trispecific Antigen-Binding Proteins

Trispecific antigen-binding proteins are purified from CHO cell culture supernatants in a two-step procedure. The constructs are subjected to affinity chromatography in a first step followed by preparative size exclusion chromatography (SEC) on Superdex 200 in a second step. Samples are buffer-exchanged and concentrated by ultrafiltration to a typical concentration of >1 mg/mL. Purity and homogeneity (typically >90%) of final samples are assessed by SDS PAGE under reducing and non-reducing conditions, followed by immunoblotting using an anti-HSA or anti idiotype antibody as well as by analytical SEC, respectively. Purified proteins are stored at aliquots at −80° C. until use.

### Example 2: Determination of Antigen Affinity by Flow Cytometry

The trispecific antigen-binding proteins of Example 1 are tested for their binding affinities to human CD16⁺ and CD20⁺ cells and cynomolgus CD16⁺ and CD20⁺ cells.

CD16⁺ and CD20⁺ cells are incubated with 100 μL of serial dilutions of the trispecific antigen-binding proteins of Example 1. After washing three times with FACS buffer the cells are incubated with 0.1 mL of 10 μg/mL mouse monoclonal anti-idiotype antibody in the same buffer for 45 min on ice. After a second washing cycle, the cells are incubated with 0.1 mL of 15 μg/mL FITC-conjugated goat anti-mouse IgG antibodies under the same conditions as before. As a control, cells are incubated with the anti-His IgG followed by the FITC-conjugated goat anti-mouse IgG antibodies without the trispecific antigen-binding proteins. The cells were then washed again and resuspended in 0.2 mL of FACS buffer containing 2 μg/mL propidium iodide (PI) in order to exclude dead cells. The fluorescence of 1×10⁴ living cells is measured using a Beckman-Coulter FC500 MPL flow cytometer using the MXP software (Beckman-Coulter, Krefeld, Germany) or a Millipore Guava EasyCyte flow cytometer using the Incyte software (Merck Millipore, Schwalbach, Germany). Mean fluorescence intensities of the cell samples are calculated using CXP software (Beckman-Coulter, Krefeld, Germany) or Incyte software (Merck Millipore, Schwalbach, Germany). After subtracting the fluorescence intensity values of the cells stained with the secondary and tertiary reagents alone the values are them used for calculation of the K_{D} values with the equation for one-site binding (hyperbola) of the GraphPad Prism (version 6.00 for Windows, GraphPad Software, La Jolla Calif. USA).

CD16 binding affinity and crossreactivity are evaluated in titration and flow cytometric experiments on CD16⁺ cells and a cynomolgus CD16⁺ cell line. CD20 binding and crossreactivity are assessed on the human CD20⁺ tumor cell lines. The K_{D} ratio of crossreactivity is calculated using the K_{D} values determined on the CHO cell lines expressing Either Recombinant Human or Recombinant Cynomolgus Antigens.

### Example 3: Cytotoxicity Assay

The trispecific antigen-binding protein of Example 1 is evaluated in vitro on its mediation of an immune response to CD20⁺ target cells.

Fluorescence labeled CD20⁺ REC-1 cells (a Mantle cell lymphoma cell line, ATCC CRL-3004) are incubated with isolated PBMC of random donors or CB15 T-cells (standardized T-cell line) as effector cells in the presence of the trispecific antigen-binding protein of Example 1. After incubation for 4 h at 37° C. in a humidified incubator, the release of the fluorescent dye from the target cells into the supernatant is determined in a spectrofluorimeter. Target cells incubated without the trispecific antigen-binding protein of Example land target cells totally lysed by the addition of saponin at the end of the incubation serve as negative and positive controls, respectively.

Based on the measured remaining living target cells, the percentage of specific cell lysis is calculated according to the following formula: [1-(number of livingtargets₍ₛₐₘₚₗₑ₎/number of living targets₍ₛₚₒₙₜₐₙₑₒᵤₛ₎]×100%. Sigmoidal dose response curves and EC₅₀ values are calculated by non-linear regression/4-parameter logistic fit using the GraphPad Software. The lysis values obtained for a given antibody concentration are used to calculate sigmoidal dose-response curves by 4 parameter logistic fit analysis using the Prism software.

### Example 4: Pharmacokinetics of Trispecific Antigen-Binding Proteins

The trispecific antigen-binding protein of Example 1 is evaluated for half-time elimination in animal studies.

The trispecific antigen-binding protein is administered to cynomolgus monkeys as a 0.5 mg/kg bolus injection intramuscularly. Another cynomolgus monkey group receives a comparable protein in size with binding domains to CD16 and CD20, but lacking HSA binding. A third and fourth group receive a protein with CD16 and HSA binding domains and a protein with CD20 and HSA binding domains respectively, and both comparable in size to the trispecific antigen-binding protein. Each test group consists of 5 monkeys. Serum samples are taken at indicated time points, serially diluted, and the concentration of the proteins is determined using a binding ELISA to CD16 and/or CD20.

Pharmacokinetic analysis is performed using the test article plasma concentrations. Group mean plasma data for each test article conforms to a multi-exponential profile when plotted against the time post-dosing. The data are fit by a standard two-compartment model with bolus input and first-order rate constants for distribution and elimination phases. The general equation for the best fit of the data for i.v. administration is: c(t)=Ae^{-αt}+Be^{-β4}, where c(t) is the plasma concentration at time t, A and B are intercepts on the Y-axis, and α and β are the apparent first-order rate constants for the distribution and elimination phases, respectively. The α-phase is the initial phase of the clearance and reflects distribution of the protein into all extracellular fluid of the animal, whereas the second or β-phase portion of the decay curve represents true plasma clearance. Methods for fitting such equations are well known in the art. For example, A=D/V(α−k21)/(α−β), B=D/V(β−k21)/(α−β), and α and β (for α>β) are roots of the quadratic equation: r²+(k12+k21+k10)r+k21k10=0 using estimated parameters of V=volume of distribution, k10=elimination rate, k12=transfer rate from compartment 1 to compartment 2 and k21=transfer rate from compartment 2 to compartment 1, and D=the administered dose.

Data analysis: Graphs of concentration versus time profiles are made using KaleidaGraph (KaleidaGraph™ V. 3.09 Copyright 1986-1997. Synergy Software. Reading, Pa.). Values reported as less than reportable (LTR) are not included in the PK analysis and are not represented graphically. Pharmacokinetic parameters are determined by compartmental analysis using WinNonlin software (WinNonlin® Professional V. 3.1 WinNonlin™ Copyright 1998-1999. Pharsight Corporation. Mountain View, Calif.). Pharmacokinetic parameters are computed as described in Ritschel W A and Kearns G L, 1999, IN: Handbook Of Basic Pharmacokinetics Including Clinical Applications, 5th edition, American Pharmaceutical Assoc., Washington, D.C.

It is expected that the trispecific antigen-binding protein of Example 1 has improved pharmacokinetic parameters such as an increase in elimination half-time as compared to proteins lacking an HSA binding domain.

### Example 5: Xenograft Tumor Model

The trispecific antigen-binding protein of Example 1 is evaluated in a xenograft model.

Female immune-deficient NOD/scid mice are sub-lethally irradiated (2 Gy) and subcutaneously inoculated with 4×10⁶ Ramos RA1 cells into their the right dorsal flank. When tumors reach 100 to 200 mm³, animals are allocated into 3 treatment groups. Groups 2 and 3 (8 animals each) are intraperitoneally injected with 1.5×10⁷ activated human T-cells. Three days later, animals from Group 3 are subsequently treated with a total of 9 intravenous doses of 50 μg trispecific antigen-binding protein of Example 1 (qdx9d). Groups 1 and 2 are only treated with vehicle. Body weight and tumor volume are determined for 30 days.

It is expected that animals treated with the trispecific antigen-binding protein of Example 1 have a statistically significant delay in tumor growth in comparison to the respective vehicle-treated control group.

### Example 6: Proof-of-Concept Clinical Trial Protocol for Administration of the Trispecific Antigen-binding Protein of Example 1 to B-cell Lymphoma Patients

This is a Phase I/II clinical trial for studying the trispecific antigen-binding protein of Example 1 as a treatment for with B-cell Lymphoma.

Study Outcomes:

Primary:

Maximum tolerated dose of trispecific antigen-binding protein of Example 1

Secondary:

To determine whether in vitro response of trispecific antigen-binding protein of Example 1 is associated with clinical response

Phase I

The maximum tolerated dose (MTD) will be determined in the phase I section of the trial.
1.1 The maximum tolerated dose (MTD) will be determined in the phase I section of the trial.
1.2 Patients who fulfill eligibility criteria will be entered into the trial to trispecific antigen-binding protein of Example 1.
1.3 The goal is to identify the highest dose of trispecific antigen-binding protein of Example 1 that can be administered safely without severe or unmanageable side effects in participants. The dose given will depend on the number of participants who have been enrolled in the study prior and how well the dose was tolerated. Not all participants will receive the same dose.

Phase II
2.1 A subsequent phase II section will be treated at the MTD with a goal of determining if therapy with therapy of trispecific antigen-binding protein of Example 1 results in at least a 20% response rate.
Primary Outcome for the Phase II—To determine if therapy of trispecific antigen-binding protein of Example 1 results in at least 20% of patients achieving a clinical response (blast response, minor response, partial response, or complete response)

Eligibility:

Histologically confirmed newly diagnosed aggressive B-cell lymphoma according to the current World Health Organisation Classification, from 2001 to 2007
Any stage of disease.
Treatment with R-CHOP or R-CHOP like regimens (+/−transplant).
Age ≥18 years
Karnofsky performance status ≥50% or ECOG performance status 0-2
Life expectancy ≥6 weeks

### Example 7: Methods to Assess Binding and Cytotoxic Activities of Trispecific Antigen Binding Molecules

Protein Production

Sequences of trispecific molecules were cloned into mammalian expression vector pCDNA 3.4 (Invitrogen) preceded by a leader sequence and followed by a 6× Histidine Tag (SEQ ID NO: 50). Expi293F cells (Life Technologies A14527) were maintained in suspension in Optimum Growth Flasks (Thomson) between 0.2 to 8×1e6 cells/ml in Expi293 media. Purified plasmid DNA was transfected into Expi293 cells in accordance with Expi293 Expression System Kit (Life Technologies, A14635) protocols, and maintained for 4-6 days post transfection. Conditioned media was partially purified by affinity and desalting chromatography. Trispecific proteins were subsequently polished by ion exchange or, alternatively, concentrated with Amicon Ultra centrifugal filtration units (EMD Millipore), applied to Superdex 200 size exclusion media (GE Healthcare) and resolved in a neutral buffer containing excipients. Fraction pooling and final purity were assessed by SDS-PAGE and analytical SEC.

Affinity Measurements

The affinities of the all binding domains molecules were measured by biolayer inferometry using an Octet instrument.

PSMA affinities were measured by loading human PSMA-Fc protein (100 nM) onto anti-human IgG Fc biosensors for 120 seconds, followed by a 60 second baseline, after which associations were measured by incubating the sensor tip in a dilution series of the trispecific molecules for 180 seconds, followed by dissociation for 50 seconds. EGFR and CD16 affinities were measured by loading human EGFR-Fc protein or human CD16-Flag-Fc protein, respectively, (100 nM) onto anti-human IgG Fc biosensors for 120 seconds, followed by a 60 second baseline, after which associations were measured by incubating the sensor tip in a dilution series of the trispecific molecules for 180 seconds, followed by dissociation for 300 seconds. Affinities to human serum albumin (HSA) were measured by loading biotinylated albumin onto streptavidin biosensors, then following the same kinetic parameters as for CD16 affinity measurements. All steps were performed at 30° C. in 0.25% casein in phosphate-buffered saline.

Cytokine Assays

AlphaLISA assays (Perkin Elmer) for TNFalpha and Interferon gamma are used to obtain evidence that innate immune cells are activated by trispecific molecules in the presence of target cells. For this assay, innate immune cells and human tumor cells are incubated in the presence of test molecules as described under cytotoxicity assays. After 48 h of incubation, 2 microliter aliquots of the assay supernatants are analyzed according to the manufacturer's instructions.

Diffusion Assays

A layer of Matrigel (75 μL) was added to 24 well Transwell inserts (0.4 μm), after which PBS was added to the upper and lower chambers (100 and 1025 μL, respectively) and equilibrated overnight at 4° C. 100 pmol of IgG or Fab (goat anti-human Fc, Jackson ImmunoResearch) or trispecific molecules was added to the upper chamber, and diffusion of each molecule into the lower chamber was quantified over time by an ELISA specific to each molecule. IgG and Fab were captured by donkey anti-goat IgG (Jackson ImmunoResearch) that had been immobilized on ELISA plates, and were detected with a horseradish peroxidase conjugated donkey anti-goat IgG (Jackson ImmunoResearch) and TMB development. Trispecific molecules were captured by human serum albumin (Athens Research & Technology) that had been immobilized on ELISA plates, and were detected with a horseradish peroxidase conjugated anti-His antibody (Genscript) and TMB development.

Relative diffusion at each timepoint was calculated as: (concentration in the lower chamber at time=t)/(concentration in the upper chamber at time=t).

Statistically significant differences in diffusion between the IgG molecule and the Fab or trispecific molecules were identified using an unpaired t-test.

### Example 8: Treatment with Trispecific Antigen-Binding Protein of Example 1 Inhibits In Vivo Tumor Growth

Murine tumor line CT26 is implanted subcutaneously in Balb/c mice and on day 7 post-implantation the average size of the tumor is measured. Mice are treated with the trispecific antigen binding protein of Example 1, or a control antibody that binds to a tumor specific target antigen but does not include an innate immune binding domain. Results show that treatment with the trispecific antigen binding protein of Example 1 inhibits tumor growth to a greater extent than the control antibody.

### Example 9: Trispecific Antigen Binding Protein of Example 1 Activates Innate Immune Cells

A population of innate immune cells, e.g., natural killer cells (NK-92) are pretreated with the trispecific antigen binding protein of Example 1, for 10 min at 4° C. and washed with FACS buffer (HBSS, 10% FBS). Levels of an activation marker for NK cells, e.g., CD56, is measured by intracellular staining, before and after incubation of the NK-92 cells with the trispecific antigen-binding protein of Example 1. Results indicate that the trispecific antigen binding protein of Example 1 is able to activate NK-92 cells, as demonstrated by increased intracellular CD56 staining.

### Example 10: Antigen Binding Protein of Example 1 Induces Activation of Dendritic Cells and Reduces Tumor Growth

C57BL/6 mice, 6-8 weeks old, are injected with 1-5×10⁴ B16 cells in the footpads. Once palpable tumor develops (5-10 days), a suitable concentration of the trispecific antigen-binding protein of Example 1 is injected subcutaneously into the contralateral limb. A control group of mice is injected with a control antibody that binds to a tumor specific target antigen but does not include an innate immune binding domain. Tumor is measured daily.

DC activation assay: Spleens and draining lymph nodes (DLN) from tumor-bearing or naive mice are harvested 3-7 days post-antigen binding protein or control treatments. Crushed spleens are digested in media containing DNAse I (Roche) and Liberase Blendzyme 2 (20,000 Mandl U/ml) (Roche). DC-enriched populations are obtained by depleting CD3 and CD19+, and gated for CD11c+ and B220+. These are evaluated by a multicolored FACS analysis using CD80, and CD86 antibodies.

Results indicate that treatment with the trispecific antigen-binding protein of Example 1 enhances the expression of DC activation markers CD80 and CD86, relative to the expression of the same in mice treated with the control antibody. Furthermore, tumor growth is seen to be reduced upon treatment with the antigen binding protein of Example 1.

TABLE 1
Sequences

| SEQ ID | | Abbrev- | |
|---|---|---|---|
| NO: | Construct | iation | Sequence |
| 1 | αEGFR:αCD16: | E:C:A | EVQLVESGGGLVQAGGSLRLSCAASGRTFSSYAMGWFRQAPGKE |
| | αAlbumin | | REFVVAINWSSGSTYYADSVKGRFTISRDNAKNTMYLQMNSLKP |
| | Trispecific | | EDTAVYYCAAGYQINSGNYNFKDYEYDYWGQGTQVTVSSGGGGS |
| | | | GGGSEVQLVESGGGLVQPGGSLKLSCAASGFTFNKYAMNWVRQA |
| | | | PGKGLEWVARIRSKYNNYATYYADSVKDRFTISRDDSKNTAYLQ |
| | | | MNNLKTEDTAVYYCVRHGNFGNSYISYWAYWGQGTLVTVSSGGG |
| | | | GSGGGGSGGGGSQDIVMTQSPDSLAVSLGERATINCKASQSVDF |
| | | | DGDSFMNWYQQKPGQPPKLLIYTTSNLESGVPDRFSGSGSGTDF |
| | | | TLTIRPLQAEDVAVYYCQQSNEDPYTFGQGTKLEIKQVTLKESG |
| | | | PALVKPTQTLTLTCTFSGFSLSTSGMGVGWIRQPPGKALEWLAH |
| | | | IWWDDDKRYNPALKSRLTISKDTSKNQVVLTMTNMDPVDTATYY |
| | | | CARINPAWFAYWGQGTLVTVSSGGGGSGGGSEVQLVESGGGLVQ |
| | | | PGNSLRLSCAASGFTFSSFGMSWVRQAPGKGLEWVSSISGSGSD |
| | | | TLYADSVKGRFTISRDNAKTTLYLQMNSLRPEDTAVYYCTIGGS |
| | | | LSRSSQGTLVTVSSHHHHHH |
| 2 | αEGFR: | E:A:C | EVQLVESGGGLVQAGGSLRLSCAASGRTFSSYAMGWFRQAPGKE |
| | αAlbumin: | | REFVVAINWSSGSTYYADSVKGRFTISRDNAKNTMYLQMNSLKP |
| | αCD16 | | EDTAVYYCAAGYQINSGNYNFKDYEYDYWGQGTQVTVSSGGGGS |
| | Trispecific | | GGGSEVQLVESGGGLVQPGNSLRLSCAASGFTESSEGMSWVRQA |
| | | | PGKGLEWVSSISGSGSDTLYADSVKGRFTISRDNAKTTLYLQMN |
| | | | SLRPEDTAVYYCTIGGSLSRSSQGTLVTVSSGGGGSGGGSEVQL |
| | | | VESGGGLVQPGGSLKLSCAASGFTFNKYAMNWVRQAPGKGLEWV |
| | | | ARIRSKYNNYATYYADSVKDRFTISRDDSKNTAYLQMNNLKTED |
| | | | TAVYYCVRHGNFGNSYISYWAYWGQGTLVTVSSGGGGSGGGGSG |
| | | | GGGSQDIVMTQSPDSLAVSLGERATINCKASQSVDFDGDSFMNW |
| | | | YQQKPGQPPKLLIYTTSNLESGVPDRFSGSGSGTDFTLTIRPLQ |
| | | | AEDVAVYYCQQSNEDPYTFGQGTKLEIKQVTLKESGPALVKPTQ |
| | | | TLTLTCTFSGFSLSTSGMGVGWIRQPPGKALEWLAHIWWDDDKR |
| | | | YNPALKSRLTISKDTSKNQVVLTMTNMDPVDTATYYCARINPAW |
| | | | FAYWGQGTLVTVSSHHHHHH |
| 3 | αAlbumin:αC | A:C:E | EVQLVESGGGLVQPGGSLKLSCAASGFTFNKYAMNWVRQAPGKG |
| | D16:αEGFR | | LEWVARIRSKYNNYATYYADSVKDRFTISRDDSKNTAYLQMNNL |
| | Trispecific | | KTEDTAVYYCVRHGNFGNSYISYWAYWGQGTLVTVSSGGGGSGG |
| | | | GGSGGGGSQDIVMTQSPDSLAVSLGERATINCKASQSVDFDGDS |
| | | | FMNWYQQKPGQPPKLLIYTTSNLESGVPDRFSGSGSGTDFTLTI |
| | | | RPLQAEDVAVYYCQQSNEDPYTFGQGTKLEIKQVTLKESGPALV |
| | | | KPTQTLTLTCTFSGFSLSTSGMGVGWIRQPPGKALEWLAHIWWD |
| | | | DDKRYNPALKSRLTISKDTSKNQVVLTMTNMDPVDTATYYCARI |
| | | | NPAWFAYWGQGTLVTVSSGGGGSGGGSEVQLVESGGGLVQAGGS |
| | | | LRLSCAASGRTESSYAMGWERQAPGKEREFVVAINWSSGSTYYA |
| | | | DSVKGRFTISRDNAKNTMYLQMNSLKPEDTAVYYCAAGYQINSG |
| | | | NYNFKDYEYDYWGQGTQVTVSSGGGGSGGGSEVQLVESGGGLVQ |
| | | | PGNSLRLSCAASGFTFSSFGMSWVRQAPGKGLEWVSSISGSGSD |
| | | | TLYADSVKGRFTISRDNAKTTLYLQMNSLRPEDTAVYYCTIGGS |
| | | | LSRSSQGTLVTVSSHHHHHH |
| 4 | αAlbumin: | A:E:C | EVQLVESGGGLVQPGNSLRLSCAASGFTFSSFGMSWVRQAPGKG |
| | αEGFR:αCD16 | | LEWVSSISGSGSDTLYADSVKGRFTISRDNAKTTLYLQMNSLRP |
| | Trispecific | | EDTAVYYCTIGGSLSRSSQGTLVTVSSGGGGSGGGSEVQLVESG |
| | | | GGLVQAGGSLRLSCAASGRTFSSYAMGWFRQAPGKEREFVVAIN |
| | | | WSSGSTYYADSVKGRFTISRDNAKNTMYLQMNSLKPEDTAVYYC |
| | | | AAGYQINSGNYNFKDYEYDYWGQGTQVTVSSGGGGSGGGSEVQL |
| | | | VESGGGLVQPGGSLKLSCAASGFTENKYAMNWVRQAPGKGLEWV |
| | | | ARIRSKYNNYATYYADSVKDRFTISRDDSKNTAYLQMNNLKTED |
| | | | TAVYYCVRHGNFGNSYISYWAYWGQGTLVTVSSGGGGSGGGGSG |
| | | | GGGSQDIVMTQSPDSLAVSLGERATINCKASQSVDFDGDSFMNW |
| | | | YQQKPGQPPKLLIYTTSNLESGVPDRFSGSGSGTDFTLTIRPLQ |
| | | | AEDVAVYYCQQSNEDPYTFGQGTKLEIKQVTLKESGPALVKPTQ |
| | | | TLTLTCTFSGFSLSTSGMGVGWIRQPPGKALEWLAHIWWDDDKR |
| | | | YNPALKSRLTISKDTSKNQVVLTMTNMDPVDTATYYCARINPAW |
| | | | FAYWGQGTLVTVSSHHHHHH |
| 5 | αCD16: | C:A:E | DIVMTQSPDSLAVSLGERATINCKASQSVDFDGDSFMNWYQQKP |
| | αAlbumin: | | GQPPKLLIYTTSNLESGVPDRFSGSGSGTDFTLTIRPLQAEDVA |
| | αEGFR | | VYYCQQSNEDPYTFGQGTKLEIKQVTLKESGPALVKPTQTLTLT |
| | Trispecific | | CTFSGFSLSTSGMGVGWIRQPPGKALEWLAHIWWDDDKRYNPAL |
| | | | KSRLTISKDTSKNQVVLTMTNMDPVDTATYYCARINPAWFAYWG |
| | | | QGTLVTVSSGGGGSGGGSEVQLVESGGGLVQPGGSLKLSCAASG |
| | | | FTENKYAMNWVRQAPGKGLEWVARIRSKYNNYATYYADSVKDRF |
| | | | TISRDDSKNTAYLQMNNLKTEDTAVYYCVRHGNFGNSYISYWAY |
| | | | WGQGTLVTVSSGGGGSGGGGSGGGGSQEVQLVESGGGLVQPGNS |
| | | | LRLSCAASGFTFSSFGMSWVRQAPGKGLEWVSSISGSGSDTLYA |
| | | | DSVKGRFTISRDNAKTTLYLQMNSLRPEDTAVYYCTIGGSLSRS |
| | | | SQGTLVTVSSGGGGSGGGSEVQLVESGGGLVQAGGSLRLSCAAS |
| | | | GRTFSSYAMGWFRQAPGKEREFVVAINWSSGSTYYADSVKGRFT |
| | | | ISRDNAKNTMYLQMNSLKPEDTAVYYCAAGYQINSGNYNFKDYE |
| | | | YDYWGQGTQVTVSSHHHHHH |
| 6 | αCD16:αEGFR: | C:E:A | DIVMTQSPDSLAVSLGERATINCKASQSVDFDGDSFMNWYQQKP |
| | αAlbumin | | GQPPKLLIYTTSNLESGVPDRFSGSGSGTDFTLTIRPLQAEDVA |
| | Trispecific | | VYYCQQSNEDPYTFGQGTKLEIKQVTLKESGPALVKPTQTLTLT |
| | | | CTFSGFSLSTSGMGVGWIRQPPGKALEWLAHIWWDDDKRYNPAL |
| | | | KSRLTISKDTSKNQVVLTMTNMDPVDTATYYCARINPAWFAYWG |
| | | | QGTLVTVSSGGGGSGGGSEVQLVESGGGLVQPGNSLRLSCAASG |
| | | | FTFSSFGMSWVRQAPGKGLEWVSSISGSGSDTLYADSVKGRFTI |
| | | | SRDNAKTTLYLQMNSLRPEDTAVYYCTIGGSLSRSSQGTLVTVS |
| | | | SGGGGSGGGSEVQLVESGGGLVQPGGSLKLSCAASGFTENKYAM |
| | | | NWVRQAPGKGLEWVARIRSKYNNYATYYADSVKDRFTISRDDSK |
| | | | NTAYLQMNNLKTEDTAVYYCVRHGNFGNSYISYWAYWGQGTLVT |
| | | | VSSGGGGSGGGGSGGGGSQEVQLVESGGGLVQAGGSLRLSCAAS |
| | | | GRTFSSYAMGWFRQAPGKEREFVVAINWSSGSTYYADSVKGRFT |
| | | | ISRDNAKNTMYLQMNSLKPEDTAVYYCAAGYQINSGNYNFKDYE |
| | | | YDYWGQGTQVTVSSHHHHHH |
| 7 | EGFR-scFv: | EGFR- | DILLTQSPVILSVSPGERVSFSCRASQSIGTNIHWYQQRTNGSP |
| | αCD16: | scFv:C:A | RLLIKYASESISGIPSRFSGSGSGTDFTLSINSVESEDIADYYC |
| | αAlbumin | | QQNNNWPTTFGAGTKLELKGGGGSGGGGSGGGGSQVQLKQSGPG |
| | | | LVQPSQSLSITCTVSGFSLTNYGVHWVRQSPGKGLEWLGVIWSG |
| | | | GNTDYNTPFTSRLSINKDNSKSQVFFKMNSLQSNDTAIYYCARA |
| | | | LTYYDYEFAYWGQGTLVTVSASGGGGSEVQLVESGGGLVQPGGS |
| | | | LKLSCAASGFTENKYAMNWVRQAPGKGLEWVARIRSKYNNYATY |
| | | | YADSVKDRFTISRDDSKNTAYLQMNNLKTEDTAVYYCVRHGNFG |
| | | | NSYISYWAYWGQGTLVTVSSGGGGSGGGGSGGGGSQDIVMTQSP |
| | | | DSLAVSLGERATINCKASQSVDFDGDSFMNWYQQKPGQPPKLLI |
| | | | YTTSNLESGVPDRFSGSGSGTDFTLTIRPLQAEDVAVYYCQQSN |
| | | | EDPYTFGQGTKLEIKQVTLKESGPALVKPTQTLTLTCTFSGFSL |
| | | | STSGMGVGWIRQPPGKALEWLAHIWWDDDKRYNPALKSRLTISK |
| | | | DTSKNQVVLTMTNMDPVDTATYYCARINPAWFAYWGQGTLVTVS |
| | | | SGGGGSGGGSEVQLVESGGGLVQPGNSLRLSCAASGFTFSSFGM |
| | | | SWVRQAPGKGLEWVSSISGSGSDTLYADSVKGRFTISRDNAKTT |
| | | | LYLQMNSLRPEDTAVYYCTIGGSLSRSSQGTLVTVSSHHHHHH |
| 8 | αPSMA:αCD16: | P:C:A | EVQLVESGGGLVQPGGSLTLSCAASRFMISEYSMHWVRQAPGKG |
| | αAlbumin | | LEWVSTINPAGTTDYAESVKGRFTISRDNAKNTLYLQMNSLKPE |
| | Trispecific | | DTAVYYCDGYGYRGQGTQVTVSSGGGGSGGGSEVQLVESGGGLV |
| | | | QPGGSLKLSCAASGFTENKYAMNWVRQAPGKGLEWVARIRSKYN |
| | | | NYATYYADSVKDRFTISRDDSKNTAYLQMNNLKTEDTAVYYCVR |
| | | | HGNFGNSYISYWAYWGQGTLVTVSSGGGGSGGGGSGGGGSQDIV |
| | | | MTQSPDSLAVSLGERATINCKASQSVDFDGDSFMNWYQQKPGQP |
| | | | PKLLIYTTSNLESGVPDRFSGSGSGTDFTLTIRPLQAEDVAVYY |
| | | | CQQSNEDPYTFGQGTKLEIKQVTLKESGPALVKPTQTLTLTCTF |
| | | | SGFSLSTSGMGVGWIRQPPGKALEWLAHIWWDDDKRYNPALKSR |
| | | | LTISKDTSKNQVVLTMTNMDPVDTATYYCARINPAWFAYWGQGT |
| | | | LVTVSSGGGGSGGGSEVQLVESGGGLVQPGNSLRLSCAASGFTF |
| | | | SSFGMSWVRQAPGKGLEWVSSISGSGSDTLYADSVKGRFTISRD |
| | | | NAKTTLYLQMNSLRPEDTAVYYCTIGGSLSRSSQGTLVTVSSHH |
| | | | HHHH |
| 9 | αPSMA: | P:A:C | EVQLVESGGGLVQPGGSLTLSCAASRFMISEYSMHWVRQAPGKG |
| | αAlbumin:αC | | LEWVSTINPAGTTDYAESVKGRFTISRDNAKNTLYLQMNSLKPE |
| | D16 | | DTAVYYCDGYGYRGQGTQVTVSSGGGGSGGGSEVQLVESGGGLV |
| | Trispecific | | QPGNSLRLSCAASGFTFSSFGMSWVRQAPGKGLEWVSSISGSGS |
| | | | DTLYADSVKGRFTISRDNAKTTLYLQMNSLRPEDTAVYYCTIGG |
| | | | SLSRSSQGTLVTVSSGGGGSGGGSEVQLVESGGGLVQPGGSLKL |
| | | | SCAASGFTENKYAMNWVRQAPGKGLEWVARIRSKYNNYATYYAD |
| | | | SVKDRFTISRDDSKNTAYLQMNNLKTEDTAVYYCVRHGNFGNSY |
| | | | ISYWAYWGQGTLVTVSSGGGGSGGGGSGGGGSQDIVMTQSPDSL |
| | | | AVSLGERATINCKASQSVDFDGDSFMNWYQQKPGQPPKLLIYTT |
| | | | SNLESGVPDRFSGSGSGTDFTLTIRPLQAEDVAVYYCQQSNEDP |
| | | | YTFGQGTKLEIKQVTLKESGPALVKPTQTLTLTCTFSGFSLSTS |
| | | | GMGVGWIRQPPGKALEWLAHIWWDDDKRYNPALKSRLTISKDTS |
| | | | KNQVVLTMTNMDPVDTATYYCARINPAWFAYWGQGTLVTVSSHH |
| | | | HHHH |
| 10 | αCD16: | C:A:P | DIVMTQSPDSLAVSLGERATINCKASQSVDFDGDSFMNWYQQKP |
| | αAlbumin: | | GQPPKLLIYTTSNLESGVPDRFSGSGSGTDFTLTIRPLQAEDVA |
| | αPSMA | | VYYCQQSNEDPYTFGQGTKLEIKQVTLKESGPALVKPTQTLTLT |
| | Trispecific | | CTFSGFSLSTSGMGVGWIRQPPGKALEWLAHIWWDDDKRYNPAL |
| | | | KSRLTISKDTSKNQVVLTMTNMDPVDTATYYCARINPAWFAYWG |
| | | | QGTLVTVSSGGGGSGGGSEVQLVESGGGLVQPGGSLKLSCAASG |
| | | | FTENKYAMNWVRQAPGKGLEWVARIRSKYNNYATYYADSVKDRF |
| | | | TISRDDSKNTAYLQMNNLKTEDTAVYYCVRHGNFGNSYISYWAY |
| | | | WGQGTLVTVSSGGGGSGGGGSGGGGSQEVQLVESGGGLVQPGNS |
| | | | LRLSCAASGFTFSSFGMSWVRQAPGKGLEWVSSISGSGSDTLYA |
| | | | DSVKGRFTISRDNAKTTLYLQMNSLRPEDTAVYYCTIGGSLSRS |
| | | | SQGTLVTVSSGGGGSGGGSEVQLVESGGGLVQPGGSLTLSCAAS |
| | | | RFMISEYSMHWVRQAPGKGLEWVSTINPAGTTDYAESVKGRFTI |
| | | | SRDNAKNTLYLQMNSLKPEDTAVYYCDGYGYRGQGTQVTVSSHH |
| | | | HHHH |
| 11 | αAlbumin: | A:P:C | EVQLVESGGGLVQPGNSLRLSCAASGFTFSSFGMSWVRQAPGKG |
| | αPSMA:αCD16 | | LEWVSSISGSGSDTLYADSVKGRFTISRDNAKTTLYLQMNSLRP |
| | Trispecific | | EDTAVYYCTIGGSLSRSSQGTLVTVSSGGGGSGGGSEVQLVESG |
| | | | GGLVQPGGSLTLSCAASRFMISEYSMHWVRQAPGKGLEWVSTIN |
| | | | PAGTTDYAESVKGRFTISRDNAKNTLYLQMNSLKPEDTAVYYCD |
| | | | GYGYRGQGTQVTVSSGGGGSEVQLVESGGGLVQPGGSLKLSCAA |
| | | | SGFTENKYAMNWVRQAPGKGLEWVARIRSKYNNYATYYADSVKD |
| | | | RFTISRDDSKNTAYLQMNNLKTEDTAVYYCVRHGNFGNSYISYW |
| | | | AYWGQGTLVTVSSGGGGSGGGGSGGGGSQDIVMTQSPDSLAVSL |
| | | | GERATINCKASQSVDFDGDSFMNWYQQKPGQPPKLLIYTTSNLE |
| | | | SGVPDRFSGSGSGTDFTLTIRPLQAEDVAVYYCQQSNEDPYTFG |
| | | | QGTKLEIKQVTLKESGPALVKPTQTLTLTCTFSGFSLSTSGMGV |
| | | | GWIRQPPGKALEWLAHIWWDDDKRYNPALKSRLTISKDTSKNQV |
| | | | VLTMTNMDPVDTATYYCARINPAWFAYWGQGTLVTVSSHHHHHH |
| 12 | αAlbumin: | A:C:P | EVQLVESGGGLVQPGNSLRLSCAASGFTFSSFGMSWVRQAPGKG |
| | αCD16:αPSMA | | LEWVSSISGSGSDTLYADSVKGRFTISRDNAKTTLYLQMNSLRP |
| | Trispecific | | EDTAVYYCTIGGSLSRSSQGTLVTVSSGGGGSGGGSDIVMTQSP |
| | | | DSLAVSLGERATINCKASQSVDFDGDSFMNWYQQKPGQPPKLLI |
| | | | YTTSNLESGVPDRFSGSGSGTDFTLTIRPLQAEDVAVYYCQQSN |
| | | | EDPYTFGQGTKLEIKQVTLKESGPALVKPTQTLTLTCTFSGFSL |
| | | | STSGMGVGWIRQPPGKALEWLAHIWWDDDKRYNPALKSRLTISK |
| | | | DTSKNQVVLTMTNMDPVDTATYYCARINPAWFAYWGQGTLVTVS |
| | | | SGGGGSGGGSEVQLVESGGGLVQPGGSLKLSCAASGFTENKYAM |
| | | | NWVRQAPGKGLEWVARIRSKYNNYATYYADSVKDRFTISRDDSK |
| | | | NTAYLQMNNLKTEDTAVYYCVRHGNFGNSYISYWAYWGQGTLVT |
| | | | VSSGGGGSGGGGSGGGGSQEVQLVESGGGLVQPGGSLTLSCAAS |
| | | | RFMISEYSMHWVRQAPGKGLEWVSTINPAGTTDYAESVKGRFTI |
| | | | SRDNAKNTLYLQMNSLKPEDTAVYYCDGYGYRGQGTQVTVSSHH |
| | | | HHHH |
| 13 | αPSMA- | PSMA- | QVQLVESGGGLVKPGESLRLSCAASGFTFSDYYMYWVRQAPGKG |
| | scFv:αCD16: | scFv:C:A | LEWVAIISDGGYYTYYSDIIKGRFTISRDNAKNSLYLQMNSLKA |
| | αAlbumin | | EDTAVYYCARGFPLLRHGAMDYWGQGTLVTVSSGGGGSGGGGSG |
| | Trispecific | | GGGSDIQMTQSPSSLSASVGDRVTITCKASQNVDTNVAWYQQKP |
| | | | GQAPKSLIYSASYRYSDVPSRFSGSASGTDFTLTISSVQSEDFA |
| | | | TYYCQQYDSYPYTFGGGTKLEIKSGGGGSEVQLVESGGGLVQPG |
| | | | GSLKLSCAASGFTENKYAMNWVRQAPGKGLEWVARIRSKYNNYA |
| | | | TYYADSVKDRFTISRDDSKNTAYLQMNNLKTEDTAVYYCVRHGN |
| | | | FGNSYISYWAYWGQGTLVTVSSGGGGSGGGGSGGGGSQDIVMTQ |
| | | | SPDSLAVSLGERATINCKASQSVDFDGDSFMNWYQQKPGQPPKL |
| | | | LIYTTSNLESGVPDRFSGSGSGTDFTLTIRPLQAEDVAVYYCQQ |
| | | | SNEDPYTFGQGTKLEIKQVTLKESGPALVKPTQTLTLTCTFSGF |
| | | | SLSTSGMGVGWIRQPPGKALEWLAHIWWDDDKRYNPALKSRLTI |
| | | | SKDTSKNQVVLTMTNMDPVDTATYYCARINPAWFAYWGQGTLVT |
| | | | VSSGGGGSGGGSEVQLVESGGGLVQPGNSLRLSCAASGFTFSSF |
| | | | GMSWVRQAPGKGLEWVSSISGSGSDTLYADSVKGRFTISRDNAK |
| | | | TTLYLQMNSLRPEDTAVYYCTIGGSLSRSSQGTLVTVSSHHHHH |
| | | | H |
| 14 | Her2- | | GVTLEVALYDYTSYNTRDLSFHKGEKFQILRMEDGVWWEARSLT |
| | Fynomer: | | TGETGYIPSNYVAPVDSIQGGGGSGGGSEVQLVESGGGLVQPGG |
| | αCD16: | | SLKLSCAASGFTENKYAMNWVRQAPGKGLEWVARIRSKYNNYAT |
| | αAlbumin | | YYADSVKDRFTISRDDSKNTAYLQMNNLKTEDTAVYYCVRHGNF |
| | Trispecific | | GNSYISYWAYWGQGTLVTVSSGGGGSGGGGSGGGGSQDIVMTQS |
| | | | PDSLAVSLGERATINCKASQSVDFDGDSFMNWYQQKPGQPPKLL |
| | | | IYTTSNLESGVPDRFSGSGSGTDFTLTIRPLQAEDVAVYYCQQS |
| | | | NEDPYTFGQGTKLEIKQVTLKESGPALVKPTQTLTLTCTFSGFS |
| | | | LSTSGMGVGWIRQPPGKALEWLAHIWWDDDKRYNPALKSRLTIS |
| | | | KDTSKNQVVLTMTNMDPVDTATYYCARINPAWFAYWGQGTLVTV |
| | | | SSGGGGSGGGSEVQLVESGGGLVQPGNSLRLSCAASGFTFSSFG |
| | | | MSWVRQAPGKGLEWVSSISGSGSDTLYADSVKGRFTISRDNAKT |
| | | | TLYLQMNSLRPEDTAVYYCTIGGSLSRSSQGTLVTVSSHHHHHH |
| 15 | αAlbumin: | A:C:M | EVQLVESGGGLVQPGGSLKLSCAASGFTFNKYAMNWVRQAPGKG |
| | αCD16: | | LEWVARIRSKYNNYATYYADSVKDRFTISRDDSKNTAYLQMNNL |
| | αMSLN | | KTEDTAVYYCVRHGNFGNSYISYWAYWGQGTLVTVSSGGGGSGG |
| | Trispecific | | GGSGGGGSQDIVMTQSPDSLAVSLGERATINCKASQSVDFDGDS |
| | | | FMNWYQQKPGQPPKLLIYTTSNLESGVPDRFSGSGSGTDFTLTI |
| | | | RPLQAEDVAVYYCQQSNEDPYTFGQGTKLEIKQVTLKESGPALV |
| | | | KPTQTLTLTCTFSGFSLSTSGMGVGWIRQPPGKALEWLAHIWWD |
| | | | DDKRYNPALKSRLTISKDTSKNQVVLTMTNMDPVDTATYYCARI |
| | | | NPAWFAYWGQGTLVTVSSGGGGSGGGSEVQLVESGGGLVQPGNS |
| | | | LRLSCAASGFTFSSFGMSWVRQAPGKGLEWVSSISGSGSDTLYA |
| | | | DSVKGRFTISRDNAKTTLYLQMNSLRPEDTAVYYCTIGGSLSRS |
| | | | SQGTLVTVSSGGGGSGGGSQVQLVQSGGGLVQPGGSLRLSCAAS |
| | | | DFDFAAYDMSWVRQAPGQGLEWVAIISHDGIDKYYDDSVKGRFT |
| | | | ISRDNSKNTLYLQMNTLRAEDTATYQCLRLGAVGQGTLVTVSSH |
| | | | HHHHH |
| 16 | αAlbumin: | A:M:C | EVQLVESGGGLVQPGNSLRLSCAASGFTFSSFGMSWVRQAPGKG |
| | αMSLN:αCD16 | | LEWVSSISGSGSDTLYADSVKGRFTISRDNAKTTLYLQMNSLRP |
| | Trispecific | | EDTAVYYCTIGGSLSRSSQGTLVTVSSGGGGSGGGSQVQLVQSG |
| | | | GGLVQPGGSLRLSCAASDFDFAAYDMSWVRQAPGQGLEWVAIIS |
| | | | HDGIDKYYDDSVKGRFTISRDNSKNTLYLQMNTLRAEDTATYQC |
| | | | LRLGAVGQGTLVTVSSGGGGSGGGSEVQLVESGGGLVQPGGSLK |
| | | | LSCAASGFTFNKYAMNWVRQAPGKGLEWVARIRSKYNNYATYYA |
| | | | DSVKDRFTISRDDSKNTAYLQMNNLKTEDTAVYYCVRHGNFGNS |
| | | | YISYWAYWGQGTLVTVSSGGGGSGGGGSGGGGSQDIVMTQSPDS |
| | | | LAVSLGERATINCKASQSVDFDGDSFMNWYQQKPGQPPKLLIYT |
| | | | TSNLESGVPDRFSGSGSGTDFTLTIRPLQAEDVAVYYCQQSNED |
| | | | PYTFGQGTKLEIKQVTLKESGPALVKPTQTLTLTCTFSGFSLST |
| | | | SGMGVGWIRQPPGKALEWLAHIWWDDDKRYNPALKSRLTISKDT |
| | | | SKNQVVLTMTNMDPVDTATYYCARINPAWFAYWGQGTLVTVSSH |
| | | | HHHHH |
| 17 | αCD16:αMSLN: | C:M:A | DIVMTQSPDSLAVSLGERATINCKASQSVDFDGDSFMNWYQQKP |
| | αAlbumin | | GQPPKLLIYTTSNLESGVPDRFSGSGSGTDFTLTIRPLQAEDVA |
| | Trispecific | | VYYCQQSNEDPYTFGQGTKLEIKQVTLKESGPALVKPTQTLTLT |
| | | | CTFSGFSLSTSGMGVGWIRQPPGKALEWLAHIWWDDDKRYNPAL |
| | | | KSRLTISKDTSKNQVVLTMTNMDPVDTATYYCARINPAWFAYWG |
| | | | QGTLVTVSSGGGGSGGGSQVQLVQSGGGLVQPGGSLRLSCAASD |
| | | | FDFAAYDMSWVRQAPGQGLEWVAIISHDGIDKYYDDSVKGRFTI |
| | | | SRDNSKNTLYLQMNTLRAEDTATYQCLRLGAVGQGTLVTVSSGG |
| | | | GGSGGGSEVQLVESGGGLVQPGGSLKLSCAASGFTFNKYAMNWV |
| | | | RQAPGKGLEWVARIRSKYNNYATYYADSVKDRFTISRDDSKNTA |
| | | | YLQMNNLKTEDTAVYYCVRHGNFGNSYISYWAYWGQGTLVTVSS |
| | | | GGGGSGGGGSGGGGSQEVQLVESGGGLVQPGNSLRLSCAASGFT |
| | | | FSSFGMSWVRQAPGKGLEWVSSISGSGSDTLYADSVKGRFTISR |
| | | | DNAKTTLYLQMNSLRPEDTAVYYCTIGGSLSRSSQGTLVTVSSH |
| | | | HHHHH |
| 18 | αAlbumin | | EVQLVESGGGLVQPGNSLRLSCAASGFTFSRFGMSWVRQAPGKG |
| | | | LEWVSSISGSGSDTLYADSVKGRFTISRDNAKTTLYLQMNSLRP |
| | | | EDTAVYYCTIGGSLSRSSQGTLVTVSS |
| 19 | αAlbumin | | EVQLVESGGGLVQPGNSLRLSCAASGFTFSKFGMSWVRQAPGKG |
| | | | LEWVSSISGSGADTLYADSLKGRFTISRDNAKTTLYLQMNSLRP |
| | | | EDTAVYYCTIGGSLSKSSQGTLVTVSS |
| 20 | αAlbumin | | EVQLVESGGGLVQPGNSLRLSCAASGFTYSSFGMSWVRQAPGKG |
| | | | LEWVSSISGSGSDTLYADSVKGRFTISRDNAKTTLYLQMNSLRP |
| | | | EDTAVYYCTIGGSLSKSSQGTLVTVSS |
| 21 | αAlbumin | | EVQLVESGGGLVQPGNSLRLSCAASGFTFSKFGMSWVRQAPGKG |
| | | | LEWVSSISGSGTDTLYADSVKGRFTISRDNAKTTLYLQMNSLRP |
| | | | EDTAVYYCTIGGSLSRSSQGTLVTVSS |
| 22 | αAlbumin | | EVQLVESGGGLVQPGNSLRLSCAASGFTESREGMSWVRQAPGKG |
| | | | LEWVSSISGSGSDTLYADSVKGRFTISRDNAKTTLYLQMNSLRP |
| | | | EDTAVYYCTIGGSLSKSSQGTLVTVSS |
| 23 | αAlbumin | | EVQLVESGGGLVQPGNSLRLSCAASGFTESKFGMSWVRQAPGKG |
| | | | LEWVSSISGSGRDTLYADSVKGRFTISRDNAKTTLYLQMNSLRP |
| | | | EDTAVYYCTIGGSLSVSSQGTLVTVSS |
| 24 | αAlbumin | | EVQLVESGGGLVQPGNSLRLSCAASGFTESSEGMSWVRQAPGKG |
| | | | LEWVSSISGSGSDTLYADSVKGRFTISRDNAKTTLYLQMNSLRP |
| | | | EDTAVYYCTIGGSLSRSSQGTLVTVSS |
| 25 | αAlbumin | | EVQLVESGGGLVQPGNSLRLSCAASGFTESREGMSWVRQAPGKG |
| | | | LEWVSSISGSGSDTLYAESVKGRFTISRDNAKTTLYLQMNSLRP |
| | | | EDTAVYYCTIGGSLSRSSQGTLVTVSS |
| 26 | αAlbumin | | EVQLVESGGGLVQPGNSLRLSCAASGFTESKFGMSWVRQAPGKG |
| | | | LEWVSSISGSGTDTLYAESVKGRFTISRDNAKTTLYLQMNSLRP |
| | | | EDTAVYYCTIGGSLSRSSQGTLVTVSS |
| 27 | αAlbumin | | EVQLVESGGGLVQPGNSLRLSCAASGFTESKFGMSWVRQAPGKG |
| | | | LEWVSSISGSGRDTLYAESVKGRFTISRDNAKTTLYLQMNSLRP |
| | | | EDTAVYYCTIGGSLSVSSQGTLVTVSS |
| 28 | αAlbumin | | EVQLLESGGGLVQPGGSLRLSCAASGFTESKYWMSWVRQAPGKG |
| | | | LEWVSSIDFMGPHTYYADSVKGRFTISRDNSKNTLYLQMNSLRA |
| | | | EDTAVYYCAKGRTSMLPMKGKFDYWGQGTLVTVSS |
| 29 | αAlbumin | | EVQLLESGGGLVQPGGSLRLSCTASGFTFDEYNMSWVRQAPGKG |
| | | | LEWVSTILPHGDRTYYADSVKGRFTISRDNSKNTLYLQMNSLRA |
| | | | EDTAVYYCAKQDPLYRFDYWGQGTLVTVSS |
| 30 | αAlbumin | | DIQMTQSPSSLSASVGDRVTITCRASQKIATYLNWYQQKPGKAP |
| | | | KLLIYRSSSLQSAVPSRFSGSGSGTVFTLTISSLQPEDFATYYC |
| | | | QQTYAVPPTFGQGTKVEIKR |
| 31 | αAlbumin | | DIQMTQSPSSLSASVGDRVTITCRASQSISSYLNWYQQKPGKAP |
| | | | KLLIYRNSPLQSGVPSRFSGSGSGTDFTLTISSLQPEDFATYYC |
| | | | QQTYRVPPTFGQGTKVEIKR |
| 32 | αAlbumin | | QVQLQESGGGLVQPGGSLRLSCEASGFTESREGMTWVRQAPGKG |
| | | | VEWVSGISSLGDSTLYADSVKGRFTISRDNAKNTLYLQMNSLKP |
| | | | EDTAVYYCTIGGSLNPGGQGTQVTVSS |
| 33 | αAlbumin | | LKEAKEKAIEELKKAGITSDYYFDLINKAKTVEGVNALKDEILK |
| | | | A |
| 34 | αAlbumin | | EVQLLESGGGLVQPGGSLRLSCAVSGIDLSNYAINWVRQAPGKC |
| | | | LEWIGIIWASGTTFYATWAKGRFTISRDNSKNTVYLQMNSLRAE |
| | | | DTAVYYCARTVPGYSTAPYFDLWGQGTLVTVSS |
| 35 | αAlbumin | | DIQMTQSPSSVSASVGDRVTITCQSSPSVWSNFLSWYQQKPGKA |
| | | | PKLLIYEASKLTSGVPSRFSGSGSGTDFTLTISSLQPEDFATYY |
| | | | CGGGYSSISDTTFGCGTKVEIKRT |
| 36 | αAlbumin | | AVQLVESGGGLVQPGNSLRLSCAASGFTERSEGMSWVRQAPGKE |
| | | | PEWVSSISGSGSDTLYADSVKGRFTISRDNAKTTLYLQMNSLKP |
| | | | EDTAVYYCTIGGSLSRSSQGTQVTVSS |
| 37 | αAlbumin | | EVQLVESGGGLVQPGNSLRLSCAASGFTESSEGMSWVRQAPGKG |
| | | | LEWVSSISGSGSDTLYADSVKGRFTISRDNAKTTLYLQMNSLRP |
| | | | EDTAVYYCTIGGSLSRSSQGTLVTVSS |
| 38 | αAlbumin | | AVQLVESGGGLVQGGGSLRLACAASERIFDLNLMGWYRQGPGNE |
| | | | RELVATCITVGDSTNYADSVKGRFTISMDYTKQTVYLHMNSLRP |
| | | | EDTGLYYCKIRRTWHSELWGQGTQVTVSS |
| 39 | αCD16 | — | DIVMTQSPDSLAVSLGERATINCKASQSVDFDGDSFMNWYQQKP |
| | | | GQPPKLLIYTTSNLESGVPDRFSGSGSGTDFTLTIRPLQAEDVA |
| | | | VYYCQQSNEDPYTFGQGTKLEIKQVTLKESGPALVKPTQTLTLT |
| | | | CTFSGFSLSTSGMGVGWIRQPPGKALEWLAHIWWDDDKRYNPAL |
| | | | KSRLTISKDTSKNQVVLTMTNMDPVDTATYYCARINPAWFAYWG |
| | | | QGTLVTVSS |
| 40 | αCD16 | — | DIVLTQSPASLAVSLGQRATISCKASQSVDFDGDSFMNWYQQKP |
| | | | GQPPKLLIYTTSNLESGIPARFSASGSGTDFTLNIHPVEEEDTA |
| | | | TYYCQQSNEDPYTFGGGTKLELKQVTLKESGPGILQPSQTLSLT |
| | | | CSFSGFSLRTSGMGVGWIRQPSGKGLEWLAHIWWDDDKRYNPAL |
| | | | KSRLTISKDTSSNQVFLKIASVDTADTATYYCAQINPAWFAYWG |
| | | | QGTLVTVSA |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A trispecific antigen-binding protein, wherein said protein comprises
(a) a first domain (A) which specifically binds to a human innate immune cell;
(b) a second domain (B) which is a half-life extension domain; and
(c) a third domain (C) which specifically binds to a target antigen,
wherein the domains are linked in the order H₂N-(A)-(C)-(B)-COOH, H₂N-(B)-(A)-(C)-COOH, H₂N-(C)-(B)-(A)-COOH, or by linkers L1 and L2.

2. The trispecific antigen-binding protein of claim 1, wherein the first domain (A) comprises a variable light chain and variable heavy chain each of which is capable of specifically binding to a human innate immune cell.

3. The trispecific antigen-binding protein of claim 2, wherein the variable light chain is a λ (lamda) light chain.

4. The trispecific antigen-binding protein of claim 2, wherein the variable light chain is a κ (kappa) light chain.

5. The trispecific antigen-binding protein of any of claims 1-4, wherein the first domain (A) comprises a single-chain variable fragment (scFv) specific to a human innate immune cell.

6. The trispecific antigen-binding protein of any of claims 1-5, wherein the first domain (A) binds to a cell surface antigen selected from CD1c, CD83, CD141, CD209, MHC II, CD123, CD303, CD304, CD16, CD56, CD1d, CD160, PLZF, NKG2d, CD94-NKG2A/C/E, CD14, CD16, CD64, CD15 CD16, 2D7 antigen, CD123, CD203c, FcεRIα, CD11b, CD193, EMR1, and Siglec-8 and activates an innate immune cell selected from dendritic cells, plasmacytoid dendritic cells, natural killer cells, natural killer T cells, monocytes, neutrophils, basophils, and eosinophils.

7. The trispecific antigen-binding protein of any one of claims 1-6, wherein the first domain (A) binds to a dendritic cell.

8. The trispecific antigen-binding protein of any one of claims 1-6, wherein the first domain (A) binds to a plasmacytoid dendritic cell.

9. The trispecific antigen-binding protein of any one of claims 1-6, wherein the first domain (A) binds to a natural killer cell.

10. The trispecific antigen-binding protein of any one of claims 1-6, wherein the first domain (A) binds to a monocyte.

11. The trispecific antigen-binding protein of any one of claims 1-6, wherein the first domain (A) binds to a neutrophil.

12. The trispecific antigen-binding protein of any one of claims 1-6, wherein the first domain (A) binds to a basophil.

13. The trispecific antigen-binding protein of any one of claims 1-6, wherein the first domain (A) binds to a eosinophil.

14. The trispecific antigen-binding protein of any of claims 1-13, wherein the first domain (A) comprises complementary determining regions (CDRs) selected from the group consisting of Lorvotuzumab, 3C12C, CSL362, 3G8, rMil2, E4, NNC141-0100.

15. The trispecific antigen-binding protein of any of claims 1-14, wherein the first domain (A) is humanized or human.

16. The trispecific antigen-binding protein of any of claims 1-15, wherein the first domain (A) has a K_{D} binding of 1000 nM or less to a cell surface marker on an innate immune cell.

17. The trispecific antigen-binding protein of any of claims 1-16, wherein the first domain (A) has a K_{D} binding of 100 nM or less a cell surface marker on an innate immune cell.

18. The trispecific antigen-binding protein of any of claims 1-17, wherein the first domain (A) has a K_{D} binding of 10 nM or less to a cell surface marker on an innate immune cell.

19. The trispecific antigen-binding protein of any of claims 1-18, wherein the first domain (A) has crossreactivity with cynomolgus innate immune cells.

20. The trispecific antigen-binding protein of any of claims 1-19, wherein the first domain (A) comprises an amino acid sequence provided herein.

21. The trispecific antigen-binding protein of any of claims 1-120, wherein the second domain (B) binds human serum albumin.

22. The trispecific antigen-binding protein of any of claims 1-21, wherein the second domain (B) comprises a scFv, a variable heavy domain (VH), a variable light domain (VL), a single domain antibody, a peptide, a ligand, or a small molecule.

23. The trispecific antigen-binding protein of any of claims 1-22, wherein the second domain (B) comprises a scFv.

24. The trispecific antigen-binding protein of any of claims 1-22, wherein the second domain (B) comprises a VH domain.

25. The trispecific antigen-binding protein of any of claims 1-22, wherein the second domain (B) comprises a VL domain.

26. The trispecific antigen-binding protein of any of claims 1-22, wherein the second domain (B) comprises a single domain antibody.

27. The trispecific antigen-binding protein of any of claims 1-22, wherein the second domain (B) comprises a peptide.

28. The trispecific antigen-binding protein of any of claims 1-22, wherein the second domain (B) comprises a ligand.

29. The trispecific antigen-binding protein of any of claims 1-22, wherein the second domain (B) comprises a small molecule entity.

30. The trispecific antigen-binding protein of any of claims 1-29, wherein the third domain (C) comprises a scFv, a VH domain, a VL domain, a non-Ig domain, a ligand, a knottin, or a small molecule entity that specifically binds to a target antigen.

31. The trispecific antigen-binding protein of any of claims 1-30, wherein the third domain (C) is specific to a cell surface molecule.

32. The trispecific antigen-binding protein of any of claims 1-31, wherein the third domain (C) is specific to a tumor antigen.

33. The trispecific antigen-binding protein of any of claims 1-32, wherein linkers L1 and L2 are peptide linkers.

34. The trispecific antigen-binding protein of claim 33, wherein linkers L1 and L2 independently consist of about 20 or less amino acid residues.

35. The trispecific antigen-binding protein of claim 34, wherein linkers L1 and L2 are each independently selected from (GS)ₙ (SEQ ID NO: 49), (GGS)ₙ (SEQ ID NO: 50), (GGGS)ₙ (SEQ ID NO: 51), (GGSG)ₙ (SEQ ID NO: 52), (GGSGG)ₙ (SEQ ID NO: 53), or (GGGGS)ₙ (SEQ ID NO: 54), wherein n is 1, 2, 3, 4, 5, 6, 7, 8, 9, or 10.

36. The trispecific antigen-binding protein of claim 35, wherein linkers L1 and L2 are each independently (GGGGS)₄ (SEQ ID NO: 55) or (GGGGS)₃ (SEQ ID NO: 56).

37. The trispecific antigen-binding protein of any of claims 1-36, wherein linkers L1 and L2 are chemical linkers.

38. The trispecific antigen-binding protein of any of claims 1-37, wherein the first domain (A) is at the N-terminus of the protein.

39. The trispecific antigen-binding protein of any of claims 1-37, wherein the second domain (B) is at the N-terminus of the protein.

40. The trispecific antigen-binding protein of any of claims 1-37, wherein the third domain (C) is at the N-terminus of the protein.

41. The trispecific antigen-binding protein of any of claims 1-37, 39 and 40, wherein the first domain (A) is at the C-terminus of the protein.

42. The trispecific antigen-binding protein of any of claims 1-38 and 40, wherein the second domain (B) is at the C-terminus of the protein.

43. The trispecific antigen-binding protein of any of claims 1-39, wherein the third domain (C) is at the C-terminus of the protein.

44. The trispecific antigen-binding protein of any of claims 1-43, wherein the protein is less than about 80 kDa.

45. The trispecific antigen-binding protein of any of claims 1-44, wherein the protein is about 50 to about 75 kDa.

46. The trispecific antigen-binding protein of any of claims 1-45, wherein the protein is less than about 50 kDa.

47. The trispecific antigen-binding protein of any of claims 1-46, wherein the protein is less than about 40 kDa.

48. The trispecific antigen-binding protein of any of claims 1-47, wherein the protein is about 20 to about 40 kDa.

49. The trispecific antigen-binding protein of any of claims 1-48, wherein the protein has an elimination half-time of at least about 50 hours.

50. The trispecific antigen-binding protein of any of claims 1-49, wherein the protein has an elimination half-time of at least about 100 hours.

51. The trispecific antigen-binding protein of any of claims 1-50, wherein the protein has increased tissue penetration as compared to an IgG to the same target antigen.

52. The trispecific antigen-binding protein of any of claims 1-51, wherein the first domain (A), second domain (B), or third domain (C) is a single domain antibody.

53. The trispecific antigen-binding protein of any of claims 1-51, wherein the first domain (A), second domain (B), and third domain (C) are a single domain antibody.

54. A trispecific antigen-binding protein, wherein said protein comprises
(a) a first domain (A) which specifically binds to a human innate immune cell;
(b) a second domain (B) which is a half-life extension domain; and
(c) a third domain (C) which specifically binds to a target antigen,
wherein the domains are linked in the order H₂N-(A)-(B)-(C)-COOH, H₂N-(A)-(C)-(B)-COOH, H₂N-(B)-(A)-(C)-COOH, H₂N-(B)-(C)-(A)-COOH, H₂N-(C)-(B)-(A)-COOH, or H₂N-(C)-(A)-(B)-COOH by linkers L1 and L2, and
wherein the first domain (A) binds to a human innate immune cell with a K_{D} of greater than 100 nM.

55. A trispecific antigen-binding protein, wherein said protein comprises
(a) a first domain (A) which specifically binds to a human innate immune cell;
(b) a second domain (B) which is a half-life extension domain; and
(c) a third domain (C) which specifically binds to a target antigen,
wherein the domains are linked in the order H₂N-(A)-(B)-(C)-COOH, H₂N-(A)-(C)-(B)-COOH, H₂N-(B)-(A)-(C)-COOH, H₂N-(B)-(C)-(A)-COOH, H₂N-(C)-(B)-(A)-COOH, or H₂N-(C)-(A)-(B)-COOH by linkers L1 and L2, and
wherein the protein has a molecular weight of less than 55 kDa.

56. A trispecific antigen-binding protein, wherein said protein comprises
(a) a first domain (A) which specifically binds to a human innate immune cell;
(b) a second domain (B) which is a half-life extension domain; and
(c) a third domain (C) which specifically binds to a target antigen,
wherein the domains are linked in the order H₂N-(A)-(B)-(C)-COOH, H₂N-(A)-(C)-(B)-COOH, H₂N-(B)-(A)-(C)-COOH, H₂N-(B)-(C)-(A)-COOH, H₂N-(C)-(B)-(A)-COOH, or H₂N-(C)-(A)-(B)-COOH by linkers L1 and L2, and
wherein the second domain (B) comprises a single domain antibody that binds to serum albumin.

57. A trispecific antigen-binding protein, wherein said protein comprises
(a) a first domain (A) which specifically binds to a human innate immune cell;
(b) a second domain (B) which is a half-life extension domain; and
(c) a third domain (C) which specifically binds to a target antigen,
wherein the domains are linked in the order H₂N-(A)-(B)-(C)-COOH, H₂N-(A)-(C)-(B)-COOH, H₂N-(B)-(A)-(C)-COOH, H₂N-(B)-(C)-(A)-COOH, H₂N-(C)-(B)-(A)-COOH, or H₂N-(C)-(A)-(B)-COOH by linkers L1 and L2, and
wherein the first domain (A), the second domain (B), and/or the third domain (C) are single domain antibodies.

58. The trispecific antigen-binding protein of any one of claims 1-57, comprising the third domain which specifically binds to the target antigen, wherein the target antigen comprises EpCAM, EGFR, HER-2, HER-3, cMet, CEA, PSMA, MSLN, or FoIR.

59. A polynucleotide encoding a trispecific antigen-binding protein diabody according to any one of claims 1 to 58.

60. A vector comprising the polynucleotide of claim 59.

61. A host cell transformed with the vector according to claim 60.

62. A pharmaceutical composition comprising (i) the trispecific antigen-binding protein according to any one of claims 1 to 58, the polynucleotide according to claim 59, the vector according to claim 60, or the host cell according to claim 61 and (ii) a pharmaceutically acceptable carrier.

63. A process for the production of a trispecific antigen-binding protein of claim 1, said process comprising culturing a host transformed or transfected with a vector comprising a nucleic acid sequence encoding a trispecific antigen-binding protein of claim 1 under conditions allowing the expression of the protein and recovering and purifying the produced protein from the culture.

64. A method for the treatment or amelioration of a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease comprising the administration of a trispecific antigen-binding protein of claim 1 to a subject in need of such a treatment or amelioration.

65. The method according to claim 64, wherein the subject is a human.

66. The method according to claim 64, wherein the method further comprises administration of an agent in combination with the trispecific antigen-binding protein of claim 1.
